(19) United States
(12) Patent Application Publication
(10) Pub. No.: US 2025/0122206 A1
(43) Pub. Date: Apr. 17, 2025

(54) PROCESS FOR THE MANUFACTURE OF A HER2 INHIBITOR

(71) Applicant: Boehringer Ingelheim International GmbH, Ingelheim am Rhein (DE)

(72) Inventors: Eugene CHONG, Ridgefield, CT (US); Weitong DONG, Temecula, CA (US); Ruoshi LI, Ridgefield, CT (US); Jason Alan MULDER, New Milford, CT (US); Alexander TROFIMOV, New Fairfield, CT (US)

(21) Appl. No.: 18/785,078

(22) Filed: Jul. 26, 2024

(30) Foreign Application Priority Data
Jul. 28, 2023 (EP) 23188330.7

(51) Int. Cl. C07D 487/04

(52) U.S. Cl. CPC C07D 487/04

(57) ABSTRACT

The present invention relates to a process for the manufacture of a HER2 inhibitor and compounds that are useful as intermediates in this process.

### FIELD OF THE INVENTION

The present invention relates to a process for the manufacture of a HER2 inhibitor and compounds that are useful as intermediates in this process.

### BACKGROUND

N-{1-[8-({3-methyl-4-[(1-methyl-1H-1,3-benzodiazol-5-yl)oxy]phenyl}amino)-[1,3]diazino[5,4-d]pyrimidin-2-yl]piperidin-4-yl}prop-2-enamide, also herein referred to as zongertinib or compound (1),

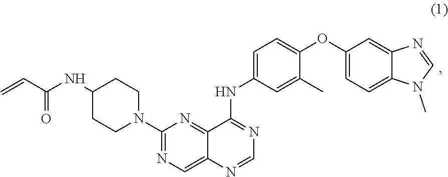 [SMILES: C=CC(=O)NC1CCN(c2ncc3ncnc(Nc4ccc(Oc5ccc6c(c5)ncn6C)c(C)c4)c3n2)CC1]

is a HER2 (ErbB2) inhibitor described in WO 2021/213800. Zongertinib is a potent and selective tyrosine kinase inhibitor of wild type and mutant HER2 that spares wild type epithelial growth factor receptor (EGFR). Therefore, it is useful for the treatment and/or prevention of diseases and/or conditions wherein the inhibition of wild type and/or mutant HER2 is of therapeutic benefit, especially oncological and/or hyperproliferative diseases, such as cancer. Compound (1) can be prepared according to a procedure described in WO 2021/213800 A1. While the process described in WO 2021/213800 can be employed to generate diverse compound libraries in an expedited way, the overall described route of synthesis comprises some specifics with respect to process controls and scalability perspectives.

The intermediates used in WO 2021/213800 comprise both nucleophilic and electrophilic functionalities. The different functionalities offer a wide variety of complex chemical possibilities, but in certain cases it might be difficult to choose the right reaction conditions, to restrict the route of synthesis to a lean product, only. Attempts to control and limit the flexible reaction might result in lower yields and the necessity to employ more costly and time extensive cleaning operations.

In addition, WO 2021/213800 also suggests the installation of an acrylamide warhead at the last step via a reaction of the corresponding amine with an acryloyl chloride. The latter may be contaminated with other reactive chlorides which are difficult to separate at reasonable costs on an industrial scale. Furthermore, highly reactive acryloyl chloride may, as a function of process conditions, result to a certain extent in reactions at other nucleophilic moieties of the molecules (e.g., secondary aniline and/or benzimidazole). Again, here the flexibility of the route of synthesis requires more strict process controls in order to avoid the processing and expensive purification of reaction mixtures, which is key in large scale processing. In addition, acryloyl chlorides are known to be highly toxic. Thus, use in large scale manufacturing is undesirable for several reasons including worker safety and environmental concerns.

These side conditions of the known process resulted in the need to develop a straightforward, repeatable and industrial scalable process that enables manufacturing of compound (1) in plant settings at reasonable costs and yields, using more benign reagents.

### BRIEF DESCRIPTION OF THE FIGURES

FIG. 1. Overview of the route of synthesis herein described. Compounds (5a) and/or (5b) are reacted either with compound (6) or compound (7) to result in compound (2).

FIG. 2. Overview of one possible route of synthesis based on a halogen leaving group.

FIG. 3. Overview of one possible route of synthesis based on a sulfur comprising leaving group.

### SUMMARY OF THE INVENTION

According to a first aspect is provided a process for the synthesis of a compound (1) according to the following formula:

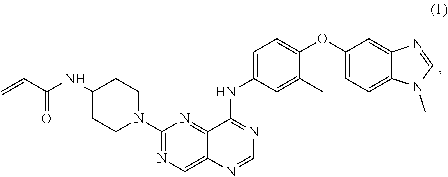 [SMILES: C=CC(=O)NC1CCN(c2ncc3ncnc(Nc4ccc(Oc5ccc6c(c5)ncn6C)c(C)c4)c3n2)CC1]

or a salt thereof, wherein the process at least comprises the process step of reacting a compound (2) according to the following formula:

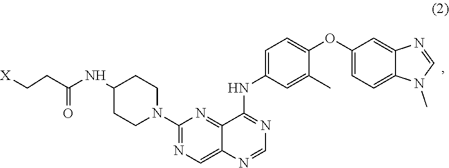

or a salt thereof, with a base in the presence of a solvent, wherein X is a leaving group or a precursor of a leaving group.

In embodiments, X is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), OC(O)C₁₋₄-alkyl, OC(O)C₁₋₄-haloalkyl, OC(O)aryl, OC(O)OC₁₋₄-alkyl, OC(O)OC₁₋₄-haloalkyl, and OC(O)Oaryl, wherein said aryl is optionally substituted by one or more substituents, each independently being the same or different C₁₋₄-alkyl.

In embodiments, X is selected from the group consisting of halogen, S(C₁₋₄-alkyl), SO(C₁₋₄-alkyl) and SO₂(C₁₋₄-alkyl).

In embodiments, compound (2) is selected from the group consisting of compounds (2a)-(2d) according to the following formulae or mixtures or salts thereof:

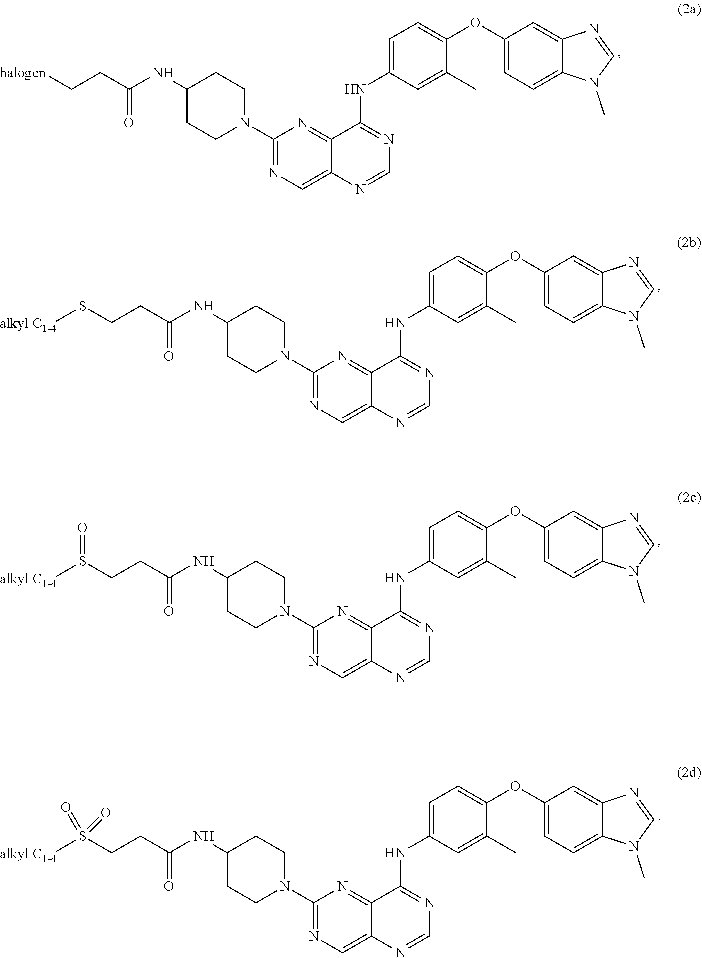

In embodiments, the solvent is selected from the group consisting of water, THF, Me-THF, MeCN, DCE, DCM, IPA, NMP, DMAc, DMF, EtOH, MTBE and mixtures thereof.

In embodiments, the solvent is selected from the group consisting of THF, MeCN, a mixture of THF and MeCN, a mixture of water and THF, a mixture of water and MeCN and a mixture of water, THF and MeCN.

In embodiments, the base is selected from the group consisting of alkaline or alkaline earth hydroxides, alkaline or alkaline earth phosphates, alkaline or alkaline earth alkoxides, superbases, alkaline or alkaline earth carbonates, aliphatic or aromatic tertiary amines and mixtures thereof.

In embodiments, the base is selected from the group consisting of KOH, NaOH, K₃PO₄, K₂CO₃, DBU, DABCO, potassium t-butoxide and Et₃N.

In embodiments, the base is potassium hydroxide.

In embodiments, X is halogen and compound (2a) is obtained by a one- or multi-step reaction comprising at least the reaction of a compound (5a) or compound (5b) according to the following formulae:

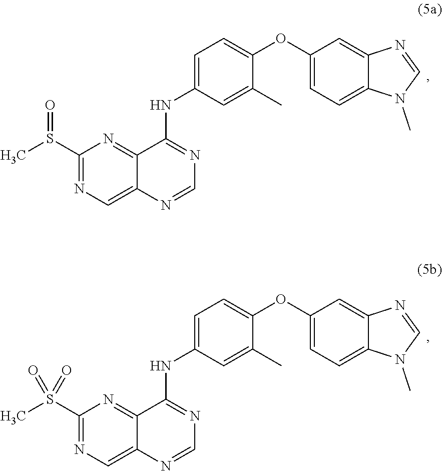 [SMILES: Cc1cc(Nc2ncnc3cnc(S(C)=O)nc23)ccc1Oc1ccc2c(c1)ncn2C ; Cc1cc(Nc2ncnc3cnc(S(C)(=O)=O)nc23)ccc1Oc1ccc2c(c1)ncn2C]

or mixtures or salts thereof and a compound (6′) according to the following formula:

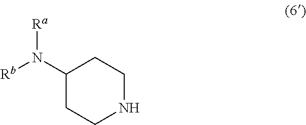

wherein R^{a} and R^{b} are each independently selected from the group consisting of hydrogen, C═O—CH₂—CH₂-halogen and a protective group (PG), wherein if both R^{a} and R^{b} are a protective group (PG), R^{a} and R^{b} together with the nitrogen atom they are bound to optionally form a ring, provided that at least one of R^{a} and R^{b} is not hydrogen, to result in compound (4b) according to the following formula:

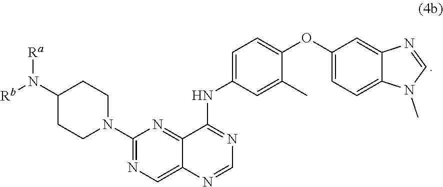

In embodiments, compound (6′) is a compound (6) according to the following formula:

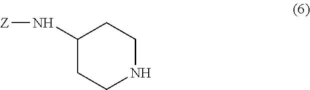

wherein Z is a protective group (PG) or C═O—CH₂—CH₂-halogen, such that the one or multi-step reaction results in compound (4) according to the following formula:

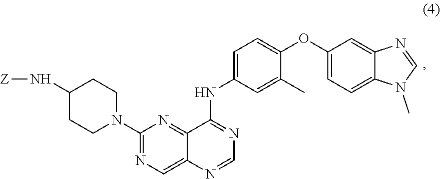

In embodiments, Z is a protective group (PG) and compound (4) is a compound (4a) according to the following formula:

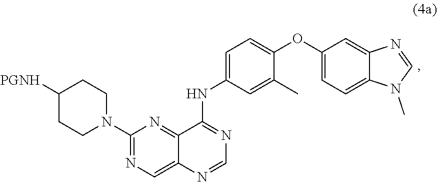

and compound (4a) is deprotected to result in compound (3) according to the following formula:

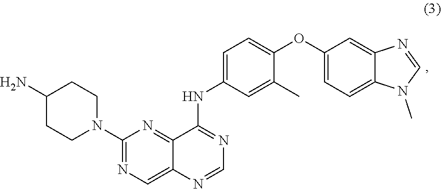 [SMILES: Cc1cc(Nc2ncnc3cnc(N4CCC(N)CC4)nc23)ccc1Oc1ccc2c(c1)ncn2C]

and compound (3) is further reacted with 3-halopropionyl halide to yield compound (2a):

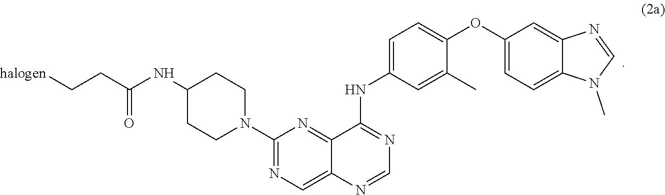

In embodiments, halogen is Cl.

In embodiments, compound (2) is obtained by a one- or multi-step reaction comprising at least the reaction of a compound (5a) or a compound (5b) according to the following formulae:

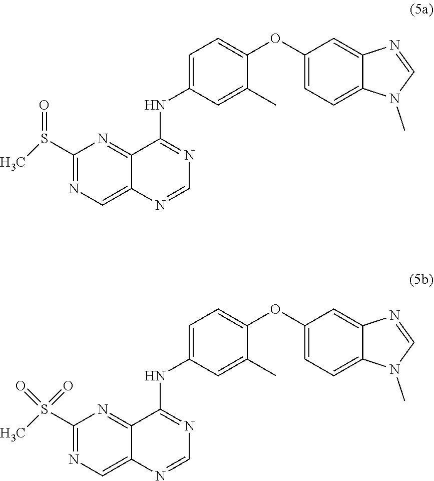 [SMILES: Cc1cc(Nc2ncnc3cnc(S(C)=O)nc23)ccc1Oc1ccc2c(c1)ncn2C ; Cc1cc(Nc2ncnc3cnc(S(C)(=O)=O)nc23)ccc1Oc1ccc2c(c1)ncn2C]

or mixtures or salts thereof and a compound (7) according to the following formula:

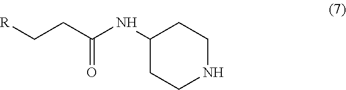

or a salt thereof, wherein R is a leaving group or the precursor of a leaving group.

In embodiments, R is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), OC(O)C₁₋₄-alkyl, OC(O)C₁₋₄-haloalkyl, OC(O)aryl, OC(O)OC₁₋₄-alkyl, OC(O)OC₁₋₄-haloalkyl, and OC(O)Oaryl, wherein said aryl is optionally substituted by one or more substituents, each independently being the same or different C₁₋₄-alkyl.

In embodiments, R comprises at least one sulfur atom.

In embodiments, R is selected from the group consisting of S(C₁₋₄-alkyl), SO(C₁₋₄-alkyl) and SO₂(C₁₋₄-alkyl).

In embodiments, compound (5a) or (5b) or mixtures or salts thereof, are reacted with a compound (7a) according to the following formula:

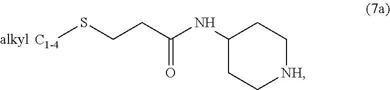

wherein the resulting reaction product compound (2b):

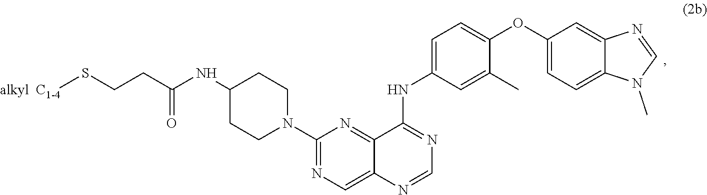

or a salt thereof is oxidized to yield compound (2c) or (2d) according to the following formulae or mixtures or salts thereof:

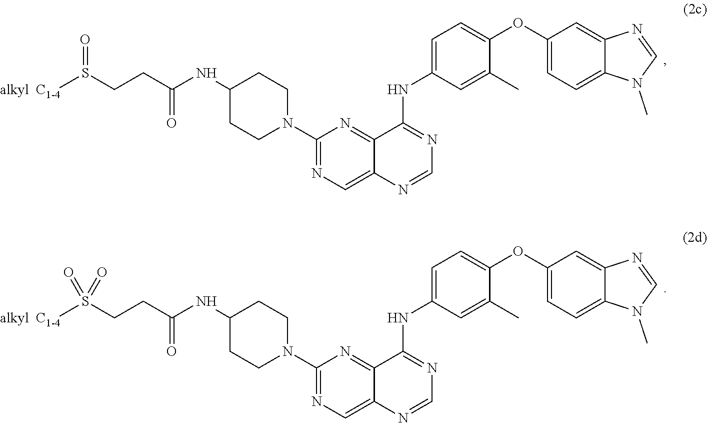

In embodiments, compound (1) is synthesized in a one-pot reaction from compound (3) or from compound (2b).

According to another aspect is provided a compound (2) according to the following formula:

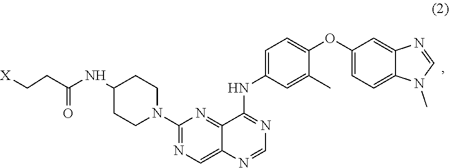

or a salt thereof, wherein X is a leaving group or a precursor of a leaving group.

In embodiments of the compound (2) or a salt thereof, X is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), OC(O)C₁₋₄-alkyl, OC(O)C₁₋₄-haloalkyl, OC(O)aryl, OC(O)OC₁₋₄-alkyl, OC(O)OC₁₋₄-haloalkyl, and OC(O)Oaryl, wherein said aryl is optionally substituted by one or more substituents, each independently being the same or different C₁₋₄-alkyl.

In embodiments of the compound (2) or a salt thereof, X is selected from the group consisting of Cl, S(CH₃), SO(CH₃) and SO₂(CH₃).

According to another aspect is provided an intermediate in the synthesis of a compound (1) according to the following formula:

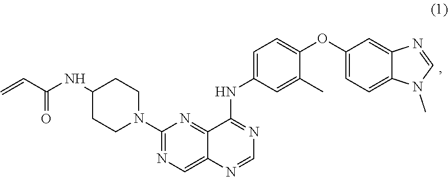 [SMILES: C=CC(=O)NC1CCN(c2ncc3ncnc(Nc4ccc(Oc5ccc6c(c5)ncn6C)c(C)c4)c3n2)CC1]

or a salt thereof, wherein the intermediate is defined above.

### DETAILED DESCRIPTION OF THE INVENTION

It is a purpose of the present invention to provide a high yield and cost-efficient process to produce compound (1). The process includes the utilization of well-defined educts, and the resulting products or intermediates are obtained in high yields and are easy to clean, if necessary at all. A further benefit of the described route of synthesis is that all process steps are up scalable and repeatable, resulting in an industrial friendly route of synthesis.

The term “C₁₋ₙ-alkyl”, wherein n is an integer selected from 2, 3 or 4, either alone or in combination with another radical, has the meaning attributed to it in the art. In particular, it denotes an acyclic, saturated, branched or linear hydrocarbon radical with 1 to n C atoms. For example, the term C₁₋₄-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂— and H₃C—C(CH₃)₂—. Preferably, C₁₋₄-alkyl is methyl.

The term halogen denotes fluorine, chlorine, bromine and iodine. Preferably, halogen is chlorine.

The term “halo” added to an “alkyl” group, i.e. haloalkyl, defines an alkyl group wherein one or more hydrogen atoms are replaced by a halogen atom selected from among fluorine, chlorine or bromine, preferably fluorine and chlorine, particularly preferred is fluorine. Examples include: H₂FC—, HF₂C—, F₃C—.

The term “aryl” as used herein, either alone or in combination with another radical, has the meaning attributed to it in the art. In particular, it denotes a carbocyclic aromatic monocyclic group containing 6 carbon atoms which is optionally further fused to a second five- or six-membered, carbocyclic group which is aromatic, saturated or unsaturated. Aryl includes, but is not limited to, phenyl, indanyl, indenyl, naphthyl, anthracenyl, phenanthrenyl, tetrahydronaphthyl and dihydronaphthyl. Preferably, aryl is phenyl.

The term “leaving group” as used herein, has the meaning attributed to it in the art. In particular, it defines an atom or substituent group (charged or uncharged) that becomes detached from a molecule in a specified reaction, wherein the molecule is considered to be the residual or main part of the substrate. Preferably, the detachment of the leaving group from the molecule enables the formation of a double bond in the molecule, wherein the position of the double bond in the molecule at least includes the atom previously bound to the leaving group. Preferably, a leaving group becomes detached in the course of an elimination reaction. Preferably, when it becomes detached, the leaving group carries a pair of electrons and thus becomes negatively charged. Leaving groups include, for example, halides (such as Br⁻, I⁻ Cl⁻, F⁻), carbonates, acetates, sulfates, sulfonyls, sulfoxyls, ethoxides and hydroxyl. When still attached to the substrate, the leaving group may be defined as an atom or group of atoms with full valence. For example, in compound (2) as defined herein, X can be a halogen leaving group, which becomes a halide when detached from compound (2).

The term “precursor of a leaving group” as used herein, has the meaning attributed to it in the art. In particular, it defines an atom or substituent group, which can be transformed in the course of a single reaction into a leaving group as defined above. A precursor of a leaving groups is especially a chemical moiety, which can be altered in the chemical composition and based on the chemical modification that group gains the ability to be (better) eliminated from the molecule during a base induced elimination reaction. The chemical reaction for the transformation of a precursor to a leaving group might, for instance, include the covalent binding of one or more electronegative atoms to the precursor. Within this reaction the ability of the group to be eliminated from the molecule might be enhanced.

The term “protective group” or “protecting group” (abbreviated PG) as used herein, has the meaning attributed to it in the art. In particular, it is a substituent or chemical moiety that is introduced into a molecule during a multi-step chemical synthesis in order to temporarily protect a certain functional group in the molecule and thus prevent an undesired reaction at this group. After the desired reaction is carried out targeting a different group in the molecule, the protecting group is again cleaved off from the molecule. The protection of a reactive group comprises a temporary chemical transformation of a reactive group into a group that does not react under conditions where the non-protected group reacts. For example, preferred protective groups include those known to mask an amine, such as Boc (tert-butoxycarbonyl), Fmoc (9-Fluorenylmethyl carbonyl), Cbz (benzyloxycarbonyl), acetamide, trifluoroacetamide, phthalimide, succinimide, benzyl, triphenylmethylamine, benzylideneamine, tosylamide, etc. Preferably, the protective group is Boc (tert-butoxycarbonyl).

The term “salt” as used herein, has the meaning attributed to it in the art. In particular, it defines a chemical compound consisting of an assembly of cations and anions. Salts include, for example, the compounds defined herein wherein one or more hydrogen atoms are replaced by one or more counterions.

The term “alkaline or alkaline earth hydroxides” as used herein, has the meaning attributed to it in the art. In particular, it defines the hydroxide salts of alkaline and alkaline earth metals. The alkaline or alkaline earth metals forming the cations may include lithium (Li), sodium (Na), potassium (K), rubidium (Rb), caesium (Cs), francium (Fr), beryllium (Be), magnesium (Mg), calcium (Ca), strontium (Sr), barium (Ba), and radium (Ra). Preferably, alkaline or alkaline earth hydroxides are selected from the group consisting of sodium hydroxide, potassium hydroxide, magnesium hydroxide and calcium hydroxide or mixtures thereof.

The term “alkaline or alkaline earth phosphates” as used herein, has the meaning attributed to it in the art. In particular, it defines the salts of an alkaline or alkaline earth metal and phosphoric acid. The phosphoric acid is present in the form of negatively charged phosphate ions and the cations are positively charged alkaline or alkaline earth metals.

The term “alkaline or alkaline earth alkoxides” as used herein, has the meaning attributed to it in the art. In particular, it defines the salts of an alkaline or alkaline earth metal and an alcoholate. The alcoholate is present in the form of negatively charged alcoholate ions and the cations are positively charged alkaline or alkaline earth metals. Preferably, the alcoholate is formed from an C1-C10 aliphatic alcohol.

The term “alkaline or alkaline earth carbonates” as used herein, has the meaning attributed to it in the art. In particular, it defines the salts of an alkaline or alkaline earth metal and carbonic acid. The carbonic acid is present in the form of negatively charged carbonate ions and the cations are positively charged alkaline or alkaline earth metals.

The term “aliphatic or aromatic tertiary amines” as used herein, has the meaning attributed to it in the art. In particular, it defines compounds formally derived from ammonia by replacing three hydrogen atoms by hydrocarbyl groups. Each hydrocarbyl group may independently comprise an aliphatic or aromatic moiety. The aliphatic moiety may be cyclic, such that the nitrogen atom of the amine is part of one or more partly or fully saturated ring(s). Multiple nitrogen atoms can be present in an aliphatic or aromatic tertiary amine. The nitrogen atom of the amine can be part of an amidine structure/moiety. Aliphatic or aromatic tertiary amines include certain superbases.

The term “one-step reaction” as used herein, has the meaning attributed to it in the art. In particular, it defines a chemical reaction, wherein only one elementary reaction is performed on one or more educts. The educts are directly transformed into one or more products, without isolating any intermediates.

The term “multi-step reaction” as used herein, has the meaning attributed to it in the art. In particular, it defines a chemical reaction, wherein more than one elementary reaction is performed on one or more educts before the desired product is obtained.

The term “one-pot reaction” as used herein, has the meaning attributed to it in the art. In particular, it defines a reaction of one or more reaction steps, wherein a reactant is subjected to successive chemical reactions in just one reactor or equivalents thereof without isolation or purification of intermediates between the chemical reactions.

Values cited herein, in particular of temperature and of reaction times, are intended as approximate. In particular, as the skilled person will appreciate, any value is associated with an error range. So, the values cited herein are intended to include all values within the error range. Unless stated otherwise, the error range is of 10%, preferably of 5%.

### Abbreviations

The following abbreviations are used herein.

| | |
|---|---|
| ° C. | degrees Celsius |
| 2-Me—THF or Me—THF | 2-methyltetrahydrofuran |
| AcCl | acetyl chloride |
| aq. | aqueous |
| Boc | tert-butoxycarbonyl |
| cat. | Catalytic |
| Cbz | benzyloxycarbonyl |
| DABCO | 1,4-diazabicyclo[2.2.2]octane |
| DBU | 1,8-diazabicyclo(5.4.0)undec-7-ene |
| DCE | dichloroethane |
| DCM | dichloromethane |
| DMF | dimethylformamide |
| DMSO | dimethyl sulfoxide |
| DMSO-d6 | deuterated dimethyl sulfoxide |
| DMAc | N,N-dimethylacetamide |
| equiv. | equivalent or equivalents |
| EtOH | ethanol |
| Et₃N | triethylamine |
| Fmoc | 9-fluorenylmethyl carbonyl |
| h, hr or hrs | hour or hours |
| HCl | hydrochloric acid |
| IPA | iso-propanol |
| IPAc | iso-propyl acetate |
| K₂CO₃ | potassium carbonate |
| K₃PO₄ | potassium phosphate tribasic |
| KF | Karl Fischer (water) titration |
| KOH | potassium hydroxide |
| LR EI MS | low resolution electron ionization mass |
| | spectroscopy |
| m, min or mins | minute or minutes |
| M | molar |
| mCPBA | meta-chloroperoxybenzoic acid |
| Me | methyl |
| MeCN or ACN | acetonitrile |
| MeOH | methanol |
| mol % | mole or moles percent |
| MTBE | methyl tert-butyl ether |
| MS | mass spectroscopy |
| NaOH | sodium hydroxide |
| NMP | N-methyl-2pyrrolidone |
| NMR | nuclear magnetic resonance |
| PG | protective group or protecting group |
| ROI | residue on ignition |
| THF | tetrahydrofuran |
| V | volume or volumes |

The process according to the invention is a process for the synthesis of a compound (1) according to the following formula:

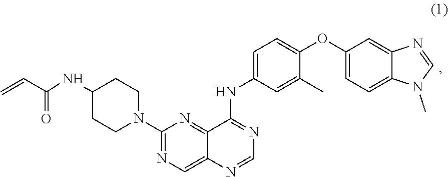 [SMILES: C=CC(=O)NC1CCN(c2ncc3ncnc(Nc4ccc(Oc5ccc6c(c5)ncn6C)c(C)c4)c3n2)CC1]

or a salt thereof, wherein the process at least comprises the process step of reacting a compound (2) according to the following formula:

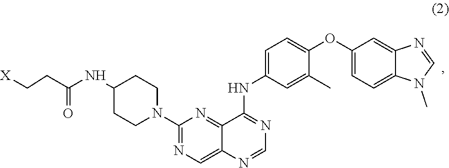

or a salt thereof, with a base in the presence of a solvent, wherein X is a leaving group or a precursor of a leaving group. The synthesis of compound (1) via an elimination reaction from compound (2), provides several advantages compared to the proposed prior art processes. Within this reaction at least a hydrogen and X are eliminated by the reaction from compound (2), resulting in the formation of a double bond at this position in compound (1). This type of reaction was not previously proposed, and it was not foreseeable, that such elimination reaction can be performed at high yields and specific at this position in the complex multicyclic heterocycle of compound (2). The overall reaction can be performed at high yields and the resulting product mixture is easy to clean. In fact, prior art processes and conditions produce more side products. Latter feature enables the superior up-scaling possibilities for this approach. Compound (1) can be present after the reaction or after the cleaning operations in the form of a neutral molecule. Furthermore, it is also possible that compound (1) is present in a charged form, thus, forming a salt with counterions. The elimination reaction is based on the presence of a leaving group or the precursor of a leaving group. Examples of leaving groups or precursors of leaving groups are given in the present description and in the examples. Preferably, X is a leaving group.

In an embodiment of the process, X is selected from the group consisting of halogen, OH, sulfur comprising leaving group (such as S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), etc.), carbonate, acetate, haloacetate (such as chloroacetate) and phosphate.

In an embodiment of the process, X is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), OC(O)C₁₋₄-alkyl, OC(O)C₁₋₄-haloalkyl, OC(O)aryl, OC(O)OC₁₋₄-alkyl, OC(O)OC₁₋₄-haloalkyl, and OC(O)Oaryl, wherein said aryl is optionally substituted by one or more substituents, each independently being the same or different C₁₋₄-alkyl.

In an embodiment of the process, X is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), and OSO₂(C₁₋₄-haloalkyl), wherein said aryl is optionally substituted by one or more substituents, each independently being the same or different C₁₋₄-alkyl. It has been found that the elimination reaction can be performed very efficiently and at high yields in cases, wherein specific X are used. The above given group for X is based on moieties comprising similar electron withdrawing functionalities, resulting in a similar and homogeneous behavior in the elimination reaction. This homogeneous behavior can be based on the similar chemistry of the heteroatoms O, S, halogen in the leaving groups or precursors of the leaving groups in the course of an elimination reaction.

Preferably OSO₂(aryl) is tosylate. As used herein, tosylate or OTs means a group with the following structure:

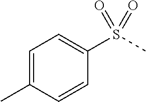

wherein . . . denotes the bond between X and the rest of compound (2).

Preferably, OSO₂(C₁₋₄-alkyl) is mesylate. As used herein, mesylate or OMs means a group with the following structure:

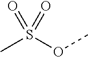

wherein . . . denotes the bond between X and the rest of compound (2).

Preferably, OSO₂(C₁₋₄-haloalkyl) is triflate. As used herein, triflate or OTf means a group with the following structure:

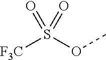

wherein . . . denotes the bond between X and the rest of compound (2).

In an embodiment of the process, X is selected from the group consisting of halogen, S(C₁₋₄-alkyl), SO(C₁₋₄-alkyl) and SO₂(C₁₋₄-alkyl). Especially the sulfur comprising groups and the halogens may be suitable to give highly efficient elimination reactions at high yields and easy to clean products.

In an embodiment of the process, X is selected from the group consisting of halogen, OH, S(CH₃), SO(CH₃), SO₂(CH₃), OSO₂(aryl), OC(O)OMe, OC(O)OCH₂CCl₃ and OC(O)O^{t}u.

In an embodiment of the process, X is selected from the group consisting of halogen, S(CH₃), SO(CH₃), SO₂(CH₃), OSO₂(aryl).

In an embodiment of the process, X is halogen or OSO₂(C₁₋₄-alkyl).

In an embodiment of the process, X is halogen or OSO₂(C₁₋₄-haloalkyl).

In an embodiment of the process, X is halogen, preferably chlorine.

In an embodiment of the process, X is S(C₁₋₄-alkyl), preferably S(CH₃).

In an embodiment of the process, X is SO(C₁₋₄-alkyl), preferably SO(CH₃).

In an embodiment of the process, X is SO₂(C₁₋₄-alkyl), preferably SO₂(CH₃).

In an embodiment of the process, compound (2) is selected from the group consisting of compounds (2a)-(2d) according to the following formulae or mixtures or salts thereof:

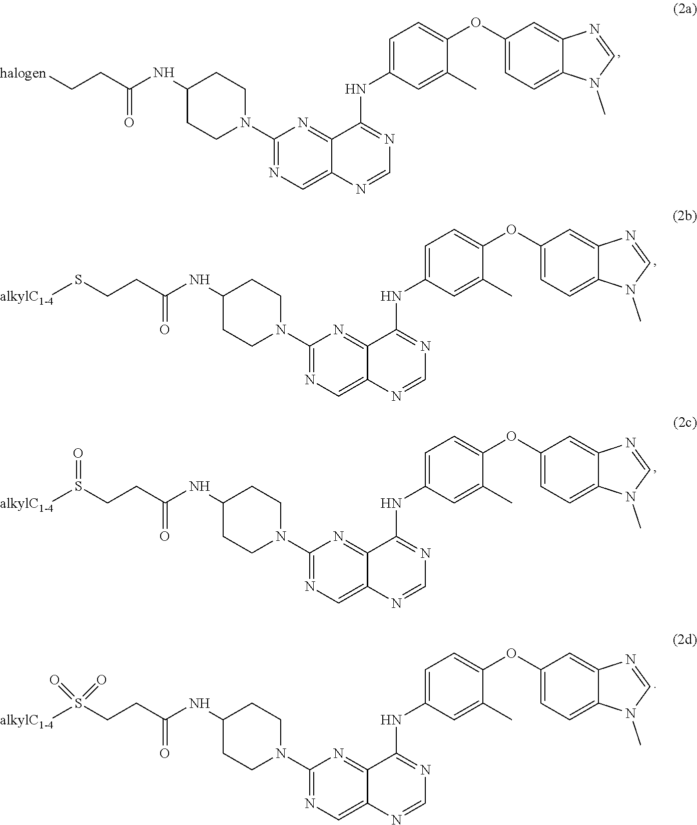

In this embodiment, halogen in compound (2a) is preferably Cl. In addition or in alternative, in this embodiment, C₁₋₄ alkyl in compounds (2b), (2c) and/or (2d) is preferably CH₃. These specific leaving groups have been found very suitable to give high yields within the course of the suggested elimination reaction. Furthermore, the resulting products are easy to clean, resulting in an efficient and easily up-scalable process.

In an embodiment, the process is a process for the synthesis of a compound (1) as defined herein or a salt thereof, wherein the process at least comprises the process step of reacting a compound (2) according to the following formula:

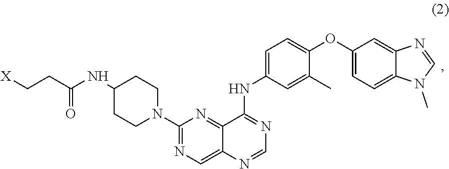

or a salt thereof, with a base in the presence of a solvent, wherein X is halogen. Preferably, halogen is selected from the group consisting of Cl, Br and I. Preferably halogen is Cl.

In an embodiment, the process is a process for the synthesis of a compound (1) as defined herein or a salt thereof, wherein the process at least comprises the process step of reacting a compound (2a) according to the following formula:

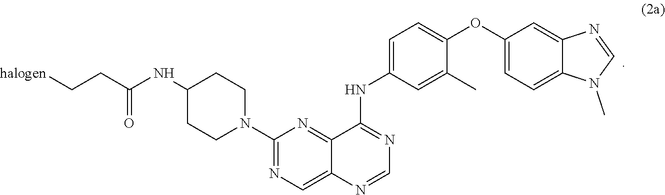

or a salt thereof, with a base in the presence of a solvent. Preferably, halogen is selected from the group consisting of Cl, Br and I. Preferably halogen is Cl. Preferably, compound (2a) is reacted in the form of a salt.

In an embodiment, the process is a process for the synthesis of a compound (1) as defined herein or a salt thereof, wherein the process at least comprises the process step of reacting a compound (2) according to the following formula:

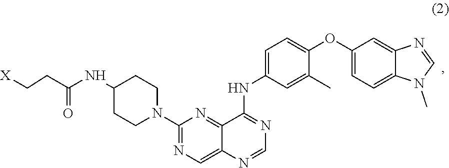

or a salt thereof, with a base in the presence of a solvent, wherein X is a sulfur comprising leaving group or precursor of a sulfur comprising leaving group. Preferably, the sulfur comprising leaving group or precursor of a sulfur comprising leaving group is selected from the group consisting of: S(C₁₋₄-alkyl), SO(C₁₋₄-alkyl), SO₂(C₁₋₄-alkyl), OSO₂aryl, OSO₂(C₁₋₄-alkyl), and OSO₂(C₁₋₄-haloalkyl).

In an embodiment, the process is a process for the synthesis of a compound (1) as defined herein or a salt thereof, wherein the process at least comprises the process step of reacting a compound (2) according to the following formula:

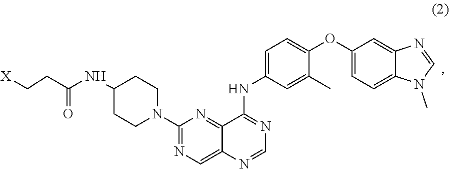

or a salt thereof, with a base in the presence of a solvent, wherein X is selected from the group consisting of S(C₁₋₄-alkyl), SO(C₁₋₄-alkyl) and SO₂(C₁₋₄-alkyl).

In an embodiment, the process is a process for the synthesis of a compound (1) as defined herein or a salt thereof, wherein the process at least comprises the process step of reacting a compound (2) according to the following formula:

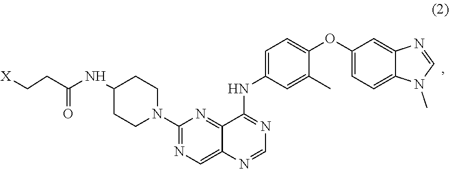

or a salt thereof, with a base in the presence of a solvent, wherein X is selected from the group consisting of S(CH₃), SO(CH₃) and SO₂(CH₃).

In an embodiment, the process is a process for the synthesis of a compound (1) as defined herein or a salt thereof, wherein the process at least comprises the process step of reacting compound (2a) or compound (2b) or compound (2c) or compound (2d) as defined above or a mixture of said compound (2c) and said compound (2d) or salts thereof with a base in the presence of a solvent. In this embodiment, halogen in compound (2a) is preferably Cl. In addition or in alternative, in this embodiment, C₁₋₄ alkyl in compounds (2b), (2c) and/or (2d) is preferably CH₃.

In an embodiment, the process is a process for the synthesis of a compound (1) as defined herein or a salt thereof, wherein the process at least comprises the process step of reacting compound (2a) or compound (2b) or compound (2c) or compound (2d) as defined above or a mixture of said compound (2c) and said compound (2d) with a base in the presence of a solvent, wherein compounds (2a), (2b), (2c), (2d) as defined above or a mixture of said compound (2c) and said compound (2d) are reacted in the form of salts. In this embodiment, halogen in compound (2a) is preferably Cl. In addition or in alternative, in this embodiment, C₁₋₄ alkyl in compounds (2b), (2c) and/or (2d) is preferably CH₃.

In an embodiment, the process is a process for the synthesis of a compound (1) as defined herein or a salt thereof, wherein the process at least comprises the process step of reacting compound (2a) or a salt thereof with a base in the presence of a solvent. In this embodiment, halogen in compound (2a) is preferably Cl.

In an embodiment, the process is a process for the synthesis of a compound (1) as defined herein or a salt thereof, wherein the process at least comprises the process step of reacting compound (2b) or a salt thereof with a base in the presence of a solvent. In this embodiment, C₁₋₄ alkyl in compound (2b) is preferably CH₃.

In an embodiment, the process is a process for the synthesis of a compound (1) as defined herein or a salt thereof, wherein the process at least comprises the process step of reacting compound (2c) or a salt thereof, with a base in the presence of a solvent. In this embodiment, C₁₋₄ alkyl in compound (2c) is preferably CH₃.

In an embodiment, the process is a process for the synthesis of a compound (1) as defined herein or a salt thereof, wherein the process at least comprises the process step of reacting compound (2d) with a base in the presence of a solvent. In this embodiment, C₁₋₄ alkyl in compounds (2d) is preferably CH₃.

In an embodiment, the process is a process for the synthesis of a compound (1) as defined herein or a salt thereof, wherein the process at least comprises the process step of reacting a mixture of compound (2c) and compound (2d) with a base in the presence of a solvent. In this embodiment, C₁₋₄ alkyl in compounds (2c) and/or (2d) is preferably CH₃.

In an embodiment of the process, the solvent is selected from the group consisting of water, THF, Me-THF, MeCN, DCE, DCM, IPA, NMP, DMAc, DMF, EtOH, MTBE and mixtures thereof. It has been found that the reaction can be performed in high yields in the selected groups of solvents. Preferably, solvent mixtures can be used.

In an embodiment of the process, the solvent is selected from the group consisting of water, THF, MeCN, DCM, IPA and mixtures thereof.

In an embodiment of the process, the solvent is selected from the group consisting of mixtures of water and at least a further solvent selected from the group consisting of THF, MeCN, DCM, IPA.

In an embodiment of the process, the solvent is a mixture of water and at least one further solvent selected from the group consisting of THF and MeCN.

In an embodiment of the process, the solvent is selected from the group consisting of: THF, MeCN, a mixture of THF and MeCN, a mixture of water and THF, a mixture of water and MeCN and a mixture of water, THF and MeCN.

In an embodiment of the process, the process comprises reacting compound (2a) or a salt thereof and the solvent is a mixture of THF and water. In this embodiment, halogen in compound (2a) is preferably Cl.

In an embodiment of the process, the process comprises reacting compound (2c), compound (2d) or salts thereof or a mixture thereof and the solvent is a mixture of water and at least one further solvent selected from the group consisting of THF and MeCN. In this embodiment, C₁₋₄ alkyl in compounds (2c) and/or (2d) is preferably CH₃.

In an embodiment of the process, the process comprises reacting compound (2c), compound (2d) or salts thereof or a mixture thereof and the solvent is a mixture of THF, MeCN and water.

In this embodiment, C₁₋₄ alkyl in compounds (2c) and/or (2d) is preferably CH₃.

In an embodiment of the process, the process comprises reacting compound (2d) or a salt thereof and the solvent is a mixture of THF, MeCN and water. In this embodiment, C₁₋₄ alkyl in compound (2d) is preferably CH₃.

In an embodiment of the process, the process comprises reacting compound (2d) or a salt thereof and the solvent is a mixture of THF, MeCN and water, wherein the volume ratio between THF and MeCN, calculated by volume THF divided by volume MeCN, is larger or equal to 0.25 and smaller or equal to 10. In this embodiment, C₁₋₄ alkyl in compound (2d) is preferably CH₃.

Any base could be used in the process according to the invention. Non-nucleophilic bases and bases other than amines are preferred. Advantageously, such bases produce fewer side products compared to nucleophilic bases and amines.

In an embodiment of the process, the base is selected from the group consisting of alkaline or alkaline earth hydroxides, alkaline or alkaline earth phosphates, alkaline or alkaline earth alkoxides, superbases (such as amidines, guanidines and phosphazenes), alkaline or alkaline earth carbonates, aliphatic or aromatic tertiary amines and mixtures thereof. These bases have been found very suitable for achieving high yields in the course of the elimination reaction. Furthermore, the bases can be easily separated, if necessary, in cleaning procedures of the product.

In an embodiment of the process, the base is selected from the group consisting of alkaline or alkaline earth hydroxides, alkaline or alkaline earth phosphates, alkaline or alkaline earth alkoxides, aliphatic or aromatic tertiary amines and mixtures thereof.

In an embodiment of the process, the base is selected from the group consisting of KOH, NaOH, K₃PO₄, K₂CO₃, DBU, DABCO, potassium t-butoxide and Et₃N.

In an embodiment of the process, the base is selected from the group consisting of KOH, NaOH, K₃PO₄, potassium t-butoxide and Et₃N.

In an embodiment of the process, the base is selected from the group consisting of KOH, NaOH and Et₃N.

In an embodiment of the process, the base is potassium hydroxide or sodium hydroxide.

In an embodiment of the process, the base is potassium hydroxide.

In an embodiment of the process, the base is potassium hydroxide and the process step from compound (2) to compound (1) is performed reacting compound (2a) or a salt thereof. In this embodiment, halogen in compound (2a) is preferably Cl.

In an embodiment of the process, the base is potassium hydroxide and the process step from compound (2) to compound (1) is performed reacting compound (2d) or a salt thereof. In this embodiment, C₁₋₄ alkyl in compound (2d) is preferably CH₃.

In an embodiment of the process, the base is potassium hydroxide and the process step from compound (2) to compound (1) is performed reacting compound (2d), compound (2c) or a mixture of compound (2c) and compound (2d) or salts thereof. In this embodiment, C₁₋₄ alkyl in compounds (2c) and/or (2d) is preferably CH₃.

In an embodiment of the process, the process step from compound (2) to compound (1) is performed in a temperature range of larger than or equal to 15° C., preferably of larger than or equal to 30° C.

In an embodiment of the process, the process step from compound (2) to compound (1) is performed in a temperature range of larger than or equal to 15° C. and smaller than or equal to 80° C., preferably of larger than or equal to 30° C. and smaller than or equal to 60° C.

In an embodiment of the process, the process step from compound (2) to compound (1) is performed in a temperature range of 30° C. to 50° C., preferably of approximately 40° C. In this embodiment, the conversion of compound (2) to compound (1) is advantageously achieved in good yields at relatively low temperatures compared to the prior art.

In an embodiment of the process, the process step from compound (2) to compound (1) is performed for a time period of larger than or equal to 5 h.

In an embodiment of the process, the process step from compound (2) to compound (1) is performed for a time period of larger than or equal to 5 h and smaller than or equal to 30 h, preferably of larger than or equal to 5 h and smaller than or equal to 25 h.

In an embodiment of the process, the process step from compound (2) to compound (1) is performed in a temperature range of larger than or equal to 15° C. for a time period of larger than or equal to 5 h.

In an embodiment of the process, the process step from compound (2) to compound (1) is performed in a temperature range of larger than or equal to 15° C. and smaller than or equal to 80° C. for a time period of larger than or equal to 5 h and smaller than or equal to 30 h.

In an embodiment of the process, the base is selected from the group consisting of KOH, NaOH and Et₃N and the process step from compound (2) to compound (1) is performed in a temperature range of larger than or equal to 15° C. and smaller than or equal to 80° C., preferably of larger than or equal to 30° C. to smaller than or equal to 60° C., preferably of approximately 40° C.

In an embodiment of the process, the base is potassium hydroxide and the process step from compound (2) to compound (1) is performed in a temperature range larger than or equal to 15° C. and smaller than or equal to 80° C., preferably of larger than or equal to 30° C. to smaller than or equal to 60° C., preferably of approximately 40° C.

In an embodiment of the process, the base is potassium hydroxide and the process step from compound (2a) to compound (1) is performed in a temperature range of larger than or equal to 15° C. and smaller than or equal to 80° C., preferably of larger than or equal to 30° C. to smaller than or equal to 60° C., preferably of approximately 40° C. In this embodiment, halogen in compound (2a) is preferably Cl.

In an embodiment of the process, the base is potassium hydroxide and the process step from compound (2d) to compound (1) is performed in a temperature range of larger than or equal to 15° C. and smaller than or equal to 80° C., preferably of larger than or equal to 30° C. to smaller than or equal to 60° C., preferably of approximately 40° C. In this embodiment, C₁₋₄ alkyl in compound (2d) is preferably CH₃.

In an embodiment of the process, the base is potassium hydroxide and the process step from compound (2c), compound (2d) or a mixture of compound (2c) and compound (2d) to compound (1) is performed in a temperature range of larger than or equal to 15° C. and smaller than or equal to 80° C., preferably of larger than or equal to 30° C. to smaller than or equal to 60° C., preferably of approximately 40° C. In this embodiment, C₁₋₄ alkyl in compounds (2c) and/or (2d) is preferably CH₃.

In an embodiment of the process, the base is selected from the group consisting of KOH, NaOH and Et₃N and the reaction from compound (2) to compound (1) is performed in a temperature range from larger than or equal to 15° C. and smaller than or equal to 80° C. for a time period of larger than or equal to 5 h and smaller than or equal to 30 h.

In an embodiment of the process, the base is potassium hydroxide and the reaction from compound (2) to compound (1) is performed in a temperature range from larger than or equal to 15° C. and smaller than or equal to 80° C. for a time period of larger than or equal to 5 h to smaller than or equal to 30 h.

In an embodiment of the process, the reaction from compound (2) to compound (1) is performed at a pH of larger than or equal to 10, preferably at a pH of larger than or equal to 11 and further preferred at a pH of larger than or equal to 12 and further preferred at a pH of larger than or equal to 13.

In an embodiment of the process, the base is selected from the group consisting of alkali or alkaline earth hydroxides or secondary or tertiary amines or mixtures thereof, and the reaction from compound (2) to compound (1) is performed at a pH of larger than or equal to 10, preferably at a pH of larger than or equal to 11 and further preferred at a pH of larger than or equal to 12 and further preferred at a pH of larger than or equal to 13.

In an embodiment of the process, the base is selected from the group consisting of alkali hydroxides or tertiary amines or mixtures thereof and the reaction from compound (2) to compound (1) is performed at a pH of larger than or equal to 10, preferably at a pH of larger than or equal to 11 and further preferred at a pH of larger than or equal to 12 and further preferred at a pH of larger than or equal to 13.

In an embodiment of the process, the base is selected from the group consisting of KOH, NaOH and Et₃N and the reaction from compound (2) to compound (1) is performed at a pH of larger than or equal to 10, preferably at a pH of larger than or equal to 11 and further preferred at a pH of larger than or equal to 13.

In an embodiment of the process, the base is Et₃N and the reaction from compound (2) to compound (1) is performed at a pH of larger than or equal to 10, preferably at a pH of larger than or equal to 11 and further preferred at a pH of larger than or equal to 13.

In an embodiment of the process, X is halogen and compound (2a) is obtained by a one- or multi-step reaction comprising at least the reaction of a compound (5a) or compound (5b) according to the following formulae:

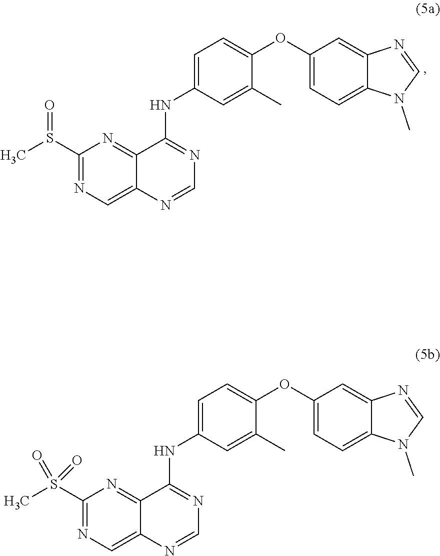 [SMILES: Cc1cc(Nc2ncnc3cnc(S(C)=O)nc23)ccc1Oc1ccc2c(c1)ncn2C ; Cc1cc(Nc2ncnc3cnc(S(C)(=O)=O)nc23)ccc1Oc1ccc2c(c1)ncn2C]

or mixtures or salts thereof and a compound (6′) according to the following formula:

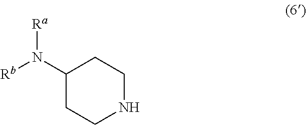

wherein R^{a} and R^{b} are each independently selected from the group consisting of hydrogen, C═O—CH₂—CH₂-halogen and a protective group (PG), wherein if both R^{a} and R^{b} are a protective group (PG), R^{a} and R^{b} together with the nitrogen atom they are bound to optionally form a ring, provided that at least one of R^{a} and R^{b} is not hydrogen, to result in compound (4b) according to the following formula:

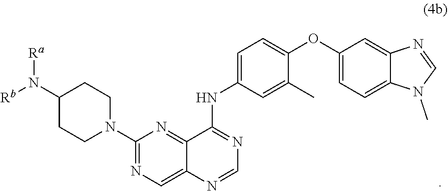

In an embodiment of the process, X is halogen and compound (2a) is obtained by a one- or multi-step reaction comprising at least the reaction of a compound (5a) or compound (5b) according to the following formulae:

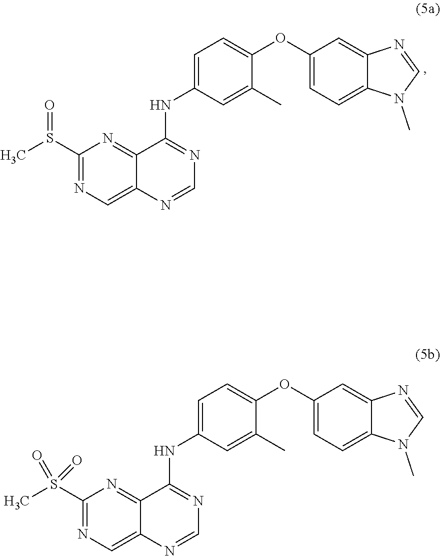 [SMILES: Cc1cc(Nc2ncnc3cnc(S(C)=O)nc23)ccc1Oc1ccc2c(c1)ncn2C ; Cc1cc(Nc2ncnc3cnc(S(C)(=O)=O)nc23)ccc1Oc1ccc2c(c1)ncn2C]

or mixtures or salts thereof and a compound (6) according to the following formula:

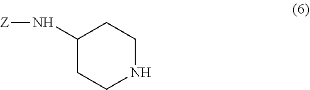

wherein Z is a protective group (PG) or C═O—CH₂—CH₂-halogen, to result in compound (4) according to the following formula:

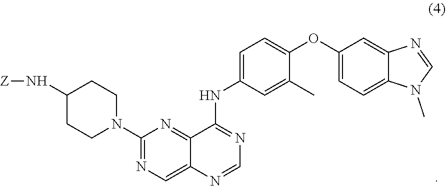

In case that the elimination reaction is based on X being a halogen it has been found suitable that the halogen comprising compound (2a) is obtained within a specific reaction sequence.

The above described reaction sequence can result in high yields and, without being bound by the theory, in a low amount of unwanted by-products, wherein the by-products may interfere with the elimination reaction to compound (1). The above-described reaction sequence includes two alternatives. On the one hand compound (4) is equivalent to compound (2a) in case that Z is C═O—CH₂—CH₂-halogen. On the other hand, in case that R^{a} and/or R^{b} or Z is a protective group (PG) further synthesis steps are necessary to result in synthesis of compound (2a). Preferably, the halogen is Cl.

In an embodiment of the process, X is halogen and compound (2a) is obtained by a one-step reaction comprising at least the reaction of a compound (5a) or compound (5b) according to the following formulae:

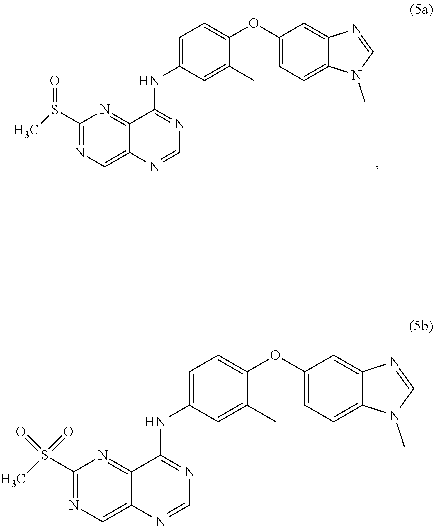 [SMILES: Cc1cc(Nc2ncnc3cnc(S(C)=O)nc23)ccc1Oc1ccc2c(c1)ncn2C ; Cc1cc(Nc2ncnc3cnc(S(C)(=O)=O)nc23)ccc1Oc1ccc2c(c1)ncn2C]

or mixtures or salts thereof and a compound (6c) according to the following formula:

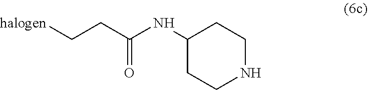

to result in compound (2a). Preferably, halogen is Cl.

In an embodiment of the process, the process step starting from compound (5a) and/or compound (5b) to compound (2a) via a reaction with compound (6c) as described above is performed in a solvent selected from the group consisting of THF, DMAc, NMP and mixtures thereof.

In an embodiment of the process, a mixture of compound (5a) and (5b) as defined is used in the one-step process, wherein said mixture comprises a larger amount of compound (5a) compared to compound (5b) in terms of percentage by weight.

In an embodiment of the process, a mixture of compound (5a) and (5b) as defined is used in the one-step process, wherein said mixture comprises compound (5a) and (5b) in a weight ratio of from 95:5 to 70:30, preferably of 85:15 to 80:20.

In an embodiment of the process, X is halogen and compound (2a) is obtained by a multi-step reaction comprising at least the reaction of a compound (5a) or compound (5b) according to the following formulae:

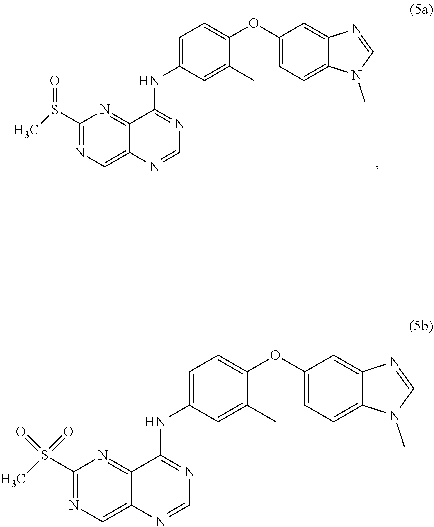 [SMILES: Cc1cc(Nc2ncnc3cnc(S(C)=O)nc23)ccc1Oc1ccc2c(c1)ncn2C ; Cc1cc(Nc2ncnc3cnc(S(C)(=O)=O)nc23)ccc1Oc1ccc2c(c1)ncn2C]

or mixtures or salts thereof and a compound (6′) according to the following formula:

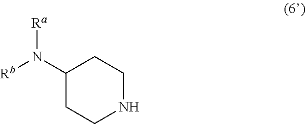

wherein R^{a} and R^{b} are each independently hydrogen or a protective group (PG), wherein R^{a} and R^{b} together with the nitrogen atom they are bound to optionally form a ring, provided that at least one of R^{a} and R^{b} is not hydrogen, to result in compound (4b) according to the following formula:

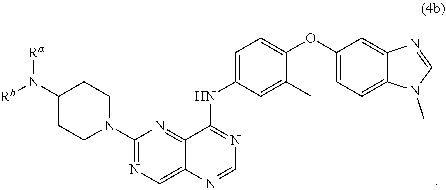

In an embodiment of the process, X is halogen and compound (2a) is obtained by a multi-step reaction comprising at least the reaction of a compound (5a) or compound (5b) according to the following formulae:

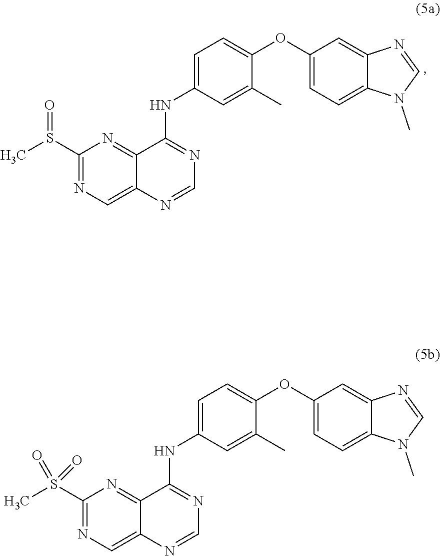 [SMILES: Cc1cc(Nc2ncnc3cnc(S(C)=O)nc23)ccc1Oc1ccc2c(c1)ncn2C ; Cc1cc(Nc2ncnc3cnc(S(C)(=O)=O)nc23)ccc1Oc1ccc2c(c1)ncn2C]

or mixtures or salts thereof and a compound (6) according to the following formula:

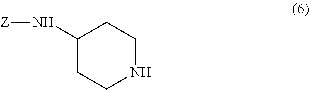

wherein Z is a protective group (PG), to result in compound (4a) according to the following

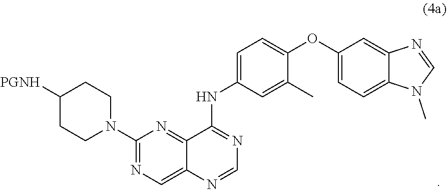

Compound (2a) can also be achieved in an alternative route, via a multi-step reaction. In this reaction sequence at first the protective group is removed from compound (4b) or (4a) and via the reaction with a 3-halopropionyl halide the halogen functional group is attached to the molecule in a second step. Preferably the 3-halopropionyl halide may at least comprise one chlorine. Preferably the 3-halopropionyl halide is 3-chloropropionyl chloride.

In an embodiment of the process, the process step starting from compound (5a) and/or compound (5b) to compound (2) via a reaction with compound (6′) or (6) as described above is performed in a solvent selected from the group consisting of THF, DMAc, NMP and mixtures thereof. In an embodiment of the process, PG is tert-butoxycarbonyl.

In an embodiment of the process, compound (6′) or (6) is a compound according to the formula (6a):

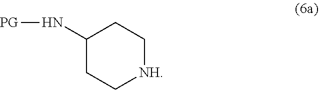

In an embodiment of the process, compound (6′) or (6) is a compound according to the formula (6b):

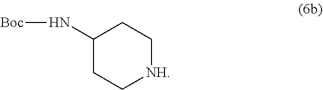 [SMILES: CC(C)(C)OC(=O)NC1CCNCC1]

In an embodiment of the process, a mixture of compound (5a) and (5b) as defined is used in the multi-step process, wherein said mixture comprises a larger amount of compound (5a) compared to compound (5b) in terms of percentage by weight.

In an embodiment of the process, a mixture of compound (5a) and (5b) as defined is used in the multi-step process, wherein said mixture comprises compound (5a) and (5b) in a weight ratio of from 95:5 to 70:30, preferably of 85:15 to 80:20.

In an embodiment of the process, X is halogen and compound (2a) is obtained by a one-step reaction comprising at least the reaction of a compound (5a) or compound (5b) or mixtures or salts thereof, wherein compound (5a) or compound (5b) are obtained by an oxidation reaction of a compound (8) or a salt thereof according to the following formula:

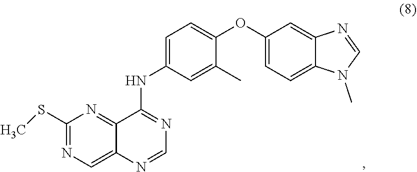 [SMILES: CSc1ncc2ncnc(Nc3ccc(Oc4ccc5c(c4)ncn5C)c(C)c3)c2n1]

and a compound (6c) to result in compound (2a).

In an embodiment of the process, X is halogen and compound (2a) is obtained by a multi-step reaction comprising at least the reaction of a compound (5a) or compound (5b) or mixtures or salts thereof, wherein compound (5a) or compound (5b) are obtained by an oxidation reaction of a compound (8) or a salt thereof, and a compound (6a) to result in compound (4a).

In an embodiment of the process, a mixture of compound (5a) and (5b) or salts thereof is obtained by an oxidation reaction of a compound (8) or a salt thereof, wherein said mixture comprises compound (5a) and (5b) in a weight ratio of from 95:5 to 70:30, preferably of 85:15 to 80:20.

In an embodiment of the process, R^{a} and R^{b} are each independently hydrogen or a protective group (PG), wherein R^{a} and R^{b} together with the nitrogen atom they are bound to optionally form a ring, provided that at least one of R^{a} and R^{b} is not hydrogen and compound (4) is a compound (4b) according to the following formula:

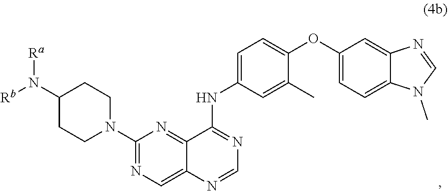

and compound (4b) is deprotected to result in compound (3) according to the following formula:

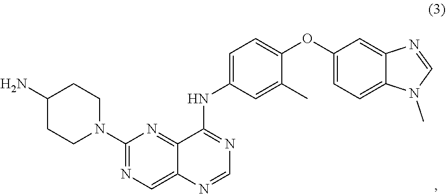 [SMILES: Cc1cc(Nc2ncnc3cnc(N4CCC(N)CC4)nc23)ccc1Oc1ccc2c(c1)ncn2C]

and compound (3) is further reacted with 3-halopropionyl halide to yield compound (2a):

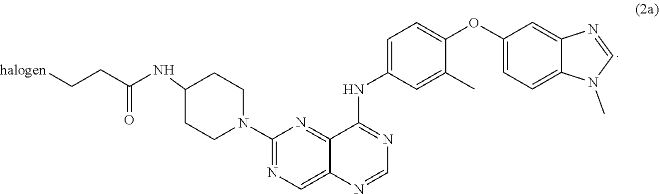

In an embodiment of the process, Z is a protective group (PG) and compound (4) is a compound (4a) according to the following formula:

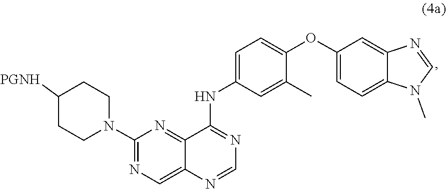

and compound (4a) is deprotected to result in compound (3) according to the following formula:

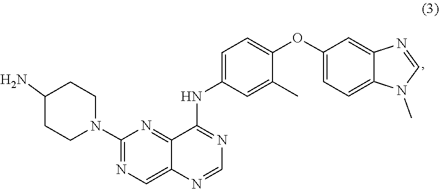 [SMILES: Cc1cc(Nc2ncnc3cnc(N4CCC(N)CC4)nc23)ccc1Oc1ccc2c(c1)ncn2C]

and compound (3) is further reacted with 3-halopropionyl halide to yield compound (2a):

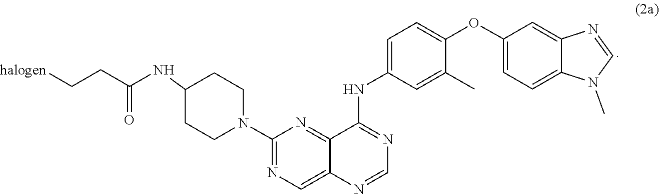

Preferably the 3-halopropionyl halide is 3-chloropropionyl chloride. Preferably, Z is tert-butoxycarbonyl (Boc) or benzyloxycarbonyl (Cbz). Preferably, the deprotection is performed in the presence of an acid. Preferably, the deprotection is performed in the presence of a solvent.

Preferably, the deprotection is performed in the presence of an acid and a solvent. Preferably, Z is tert-butoxycarbonyl or benzyloxycarbonyl and the deprotection is performed in the presence of an acid. Preferably, Z is tert-butoxycarbonyl or benzyloxycarbonyl and the deprotection is performed in the presence of an acid and a solvent. Preferably, the deprotection is performed in the presence of a solvent at a temperature larger than or equal to 50° C. and smaller than or equal to 75° C. Preferably, the deprotection is performed in the presence of an acid and a solvent at a temperature larger than or equal to 50° C. and smaller than or equal to 75° C. Preferably, Z is tert-butoxycarbonyl or benzyloxycarbonyl and the deprotection is performed in the presence of an acid at a temperature larger than or equal to 50° C. and smaller than or equal to 75° C. Preferably, Z is tert-butoxycarbonyl or benzyloxycarbonyl and the deprotection is performed in the presence of an acid and a solvent at a temperature larger than or equal to 50° C. and smaller than or equal to 75° C. Preferably, the solvent in the de-protection is an alcohol. Preferably the solvent is IPA. Preferably, the acid is selected from the group consisting of hydrochloric acid, trifluoric acid, acyl chloride and sulfuric acid. Preferably, the second reaction step including the reaction with the 3-halopropionyl halide is performed in the presence of a solvent and the solvent is selected from the group consisting of DMSO, MeCN, THF, water and any mixture thereof. Preferably, the second reaction step including the reaction with the 3-halopropionyl halide is performed in the presence of a solvent and the solvent is a solvent mixture at least comprising water and THF.

In an embodiment of the process, halogen is Cl. High yield elimination reactions can be achieved by using chlorine as the leaving halogen. Besides the high yield also easy and low cost cleaning operations can be used in order to isolate compound (1), if necessary at all.

In an embodiment of the process, compound (1) is synthesized in a one-pot reaction from compound (3) or from compound (2b). An additional advantage of the proposed route of synthesis is, that several synthesis steps can be performed in a common reaction surrounding, i.e. one-pot. Within the one-pot set-up it is not necessary to isolate the intermediates prior to a next reaction step. In the case of compound (2b) the oxidation reaction at the sulfur and the elimination reaction can be performed in one reaction surrounding. In the case of compound (3) the attachment of the halogen comprising moiety and the elimination of the halogen can be performed in the same chemical surrounding.

In an embodiment of the process, compound (2) is obtained by a one- or multi-step reaction comprising at least the reaction of a compound (5a) or a compound (5b) according to the following formulae:

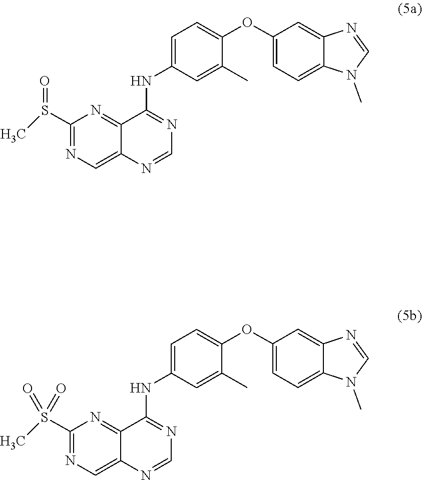 [SMILES: Cc1cc(Nc2ncnc3cnc(S(C)=O)nc23)ccc1Oc1ccc2c(c1)ncn2C ; Cc1cc(Nc2ncnc3cnc(S(C)(=O)=O)nc23)ccc1Oc1ccc2c(c1)ncn2C]

or mixtures or salts thereof and a compound (7) according to the following formula:

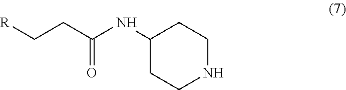

or a salt thereof, wherein R is a leaving group or the precursor of a leaving group. Preferably, in this embodiment, R and thus X comprise at least one sulfur atom. High yields with respect to the synthesis of compound (2) can be achieved in cases, wherein compound (7) is either attached to compound (5a) or (5b).

In an embodiment of the process, X comprises at least one sulfur atom and compound (2) is obtained by a one- or multi-step reaction comprising at least the reaction of a compound (5a) or a compound (5b) according to the following formulae:

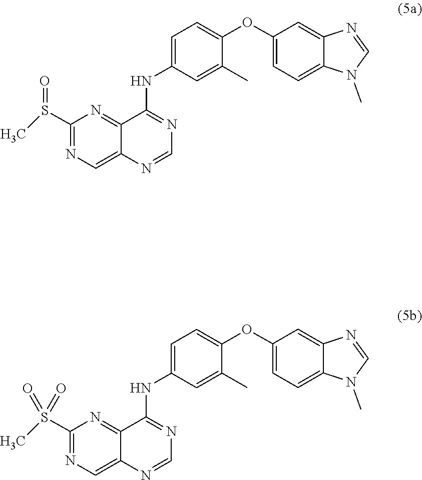 [SMILES: Cc1cc(Nc2ncnc3cnc(S(C)=O)nc23)ccc1Oc1ccc2c(c1)ncn2C ; Cc1cc(Nc2ncnc3cnc(S(C)(=O)=O)nc23)ccc1Oc1ccc2c(c1)ncn2C]

or mixtures or salts thereof and a compound (7) according to the following formula:

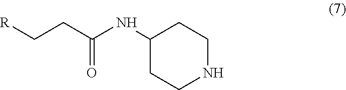

or a salt thereof, wherein R is selected from the group consisting of S(C₁₋₄-alkyl), SO(C₁₋₄-alkyl), SO₂(C₁₋₄-alkyl), OSO₂aryl, OSO₂(C₁₋₄-alkyl) and OSO₂(C₁₋₄-haloalkyl), wherein said aryl is optionally substituted by one or more substituents, each independently being the same or different C₁₋₄-alkyl.

In an embodiment of the process, R is selected from the group consisting of OH, sulfur comprising leaving group (such as S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), etc.), carbonate, acetate, haloacetate (such as chloroacetate) and phosphate.

In an embodiment of the process, R is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), OC(O)C₁ 4-alkyl, OC(O)C₁ 4-haloalkyl, OC(O)aryl, OC(O)OC₁₋₄-alkyl, OC(O)OC₁₋₄-haloalkyl, and OC(O)Oaryl, wherein said aryl is optionally substituted by one or more substituents, each independently being the same or different C₁₋₄-alkyl.

In an embodiment of the process, R is selected from the group consisting of S(C₁₋₄-alkyl), SO(C₁₋₄-alkyl), SO₂(C₁₋₄-alkyl), OSO₂-aryl, OSO₂(C₁₋₄-alkyl) and OSO₂(C₁₋₄-haloalkyl), wherein said aryl is optionally substituted by one or more substituents, each independently being the same or different C₁₋₄-alkyl.

In an embodiment of the process, R comprises at least one sulfur atom.

In an embodiment of the process, R is selected from the group consisting of S(C₁₋₄-alkyl), SO(C₁₋₄-alkyl) and SO₂(C₁₋₄-alkyl).

In an embodiment of the process, R is S(C₁₋₄-alkyl).

In an embodiment of the process, X comprises at least one sulfur atom and compound (2) is obtained by a multi-step reaction comprising at least an oxidation reaction of a compound (8) or a salt thereof according to the following formula:

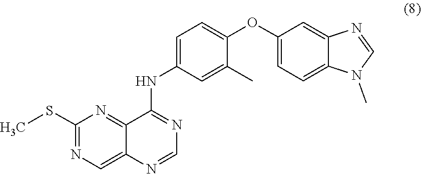 [SMILES: CSc1ncc2ncnc(Nc3ccc(Oc4ccc5c(c4)ncn5C)c(C)c3)c2n1]

to result in compound (5a) or a compound (5b) as defined herein or mixtures or salts thereof and a further reaction of compound (5a) or compound (5b) with a compound (7) according to the following formula:

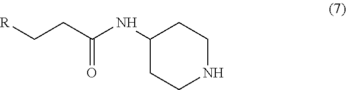

or a salt thereof, wherein R is selected from the group consisting of S(C₁₋₄-alkyl), SO(C₁₋₄-alkyl), SO₂(C₁₋₄-alkyl), OSO₂-aryl, OSO₂(C₁₋₄-alkyl) and OSO₂(C₁₋₄-haloalkyl), wherein said aryl is optionally substituted by one or more substituents, each independently being the same or different C₁₋₄-alkyl. Preferably, a hydrochloride salt of compound (8) is used in the first step. Preferably, a monohydrochloride or dihydrochloride salt of compound (8) is used. Preferably, the oxidizing agent is H₂O₂. Preferably, a mixture of compound (5a) and (5b) is obtained, wherein said mixture comprises a larger amount of compound (5a) compared to compound (5b) in terms of percentage by weight. Preferably, said mixture comprises compound (5a) and (5b) in a weight ratio of from 95:5 to 70:30, preferably of 85:15 to 80:20.

In an embodiment of the process, X comprises at least one sulfur atom and compound (2) is obtained by a one- or multi-step reaction comprising at least the reaction of a compound (5a) or a compound (5b) as defined herein or mixtures or salts thereof and a compound (7) according to the following formula:

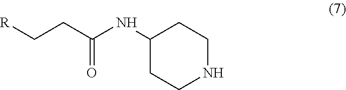

or a salt thereof in the presence of a solvent, wherein R is selected from the group consisting of S(C₁₋₄-alkyl), SO(C₁₋₄-alkyl), SO₂(C₁₋₄-alkyl), OSO₂-aryl, OSO₂(C₁₋₄-alkyl) and OSO₂(C₁₋₄-haloalkyl), wherein said aryl is optionally substituted by one or more substituents, each independently being the same or different C₁₋₄-alkyl. Preferably, the solvent is organic and non-nucleophilic. Preferably, the solvent is 2-Me-THF. Preferably, this reaction step is conducted under heating. Preferably, the reaction is performed at a temperature larger than 50° C. and lower or equal to 70° C. Preferably, the hydrochloride salt of compound (7) is used. Preferably, compound (7) can be used in the form of a monohydrochloride or dihydrochloride salt.

In a preferred embodiment, X comprises at least one sulfur atom and compound (2) is obtained by a one-step reaction comprising at least the reaction of a compound (5a) or a compound (5b) as defined herein or mixtures or salts thereof and a compound (7) according to the following formula:

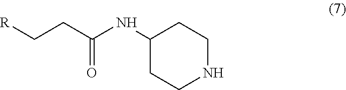

or a salt thereof in the presence of a solvent, wherein R is selected from the group consisting of S(C₁₋₄-alkyl), SO(C₁₋₄-alkyl), SO₂(C₁₋₄-alkyl), OSO₂-aryl, OSO₂(C₁₋₄-alkyl) and OSO₂(C₁₋₄-haloalkyl), wherein said aryl is optionally substituted by one or more substituents, each independently being the same or different C₁₋₄-alkyl. Preferably, R is selected from the group consisting of SO(C₁₋₄-alkyl), SO₂(C₁₋₄-alkyl), OSO₂-aryl, OSO₂(C₁₋₄-alkyl) and OSO₂(C₁₋₄-haloalkyl). Preferably, the solvent is organic and non-nucleophilic. Preferably, the solvent is 2-Me-THF. Preferably, this reaction step is conducted under heating. Preferably, the reaction is performed at a temperature larger than 50° C. and lower or equal to 70° C. Preferably, the hydrochloride salt of compound (7) is used. Preferably, compound (7) can be used in the form of a monohydrochloride or dihydrochloride salt.

In an embodiment of the process, X comprises at least one sulfur atom and compound (2) is obtained by a multi-step reaction comprising at least an oxidation reaction of a compound (8) or a salt thereof according to the following formula:

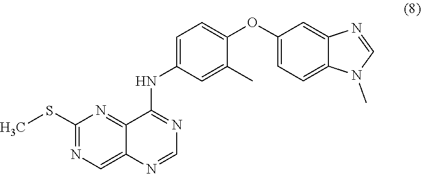 [SMILES: CSc1ncc2ncnc(Nc3ccc(Oc4ccc5c(c4)ncn5C)c(C)c3)c2n1]

to result in compound (5a) or a compound (5b) as defined herein or mixtures or salts thereof and a further reaction of compound (5a) or compound (5b) with a compound (7) according to the following formula:

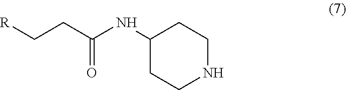

or a salt thereof in the presence of a solvent, wherein R is selected from the group consisting of S(C₁₋₄-alkyl), SO(C₁₋₄-alkyl), SO₂(C₁₋₄-alkyl), OSO₂-aryl, OSO₂(C₁₋₄-alkyl), OSO₂(C₁₋₄-haloalkyl), wherein said aryl is optionally substituted by one or more substituents, each independently being the same or different C₁₋₄-alkyl. Preferably, a hydrochloride salt of compound (8) is used in the first step. Preferably, a monohydrochloride or dihydrochloride salt of compound (8) is used. Preferably, the oxidizing agent is H₂O₂. Preferably, a mixture of compound (5a) and (5b) is obtained, wherein said mixture comprises a larger amount of compound (5a) compared to compound (5b) in terms of percentage by weight. Preferably, said mixture comprises compound (5a) and (5b) in a weight ratio of from 95:5 to 70:30, preferably of 85:15 to 80:20. Preferably, the solvent is organic and non-nucleophilic. Preferably, the solvent is 2-Me-THF. Preferably, this reaction step is conducted under heating. Preferably, the reaction is performed at a temperature larger than 50° C. and lower or equal to 70° C. Preferably, the hydrochloride salt of compound (7) is used. Preferably, compound (7) can be used in the form of a monohydrochloride or dihydrochloride salt.

In an embodiment of the process, compounds (5a) or (5b) or mixtures or salts thereof, are reacted with a compound (7a) according to the following formula:

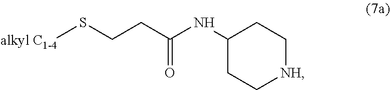

wherein the resulting reaction product compound (2b):

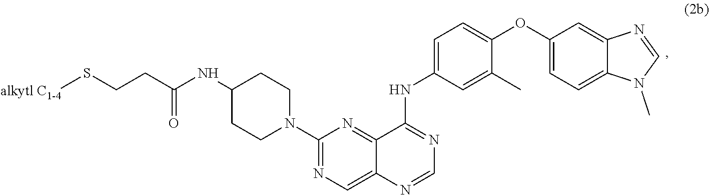

or a salt thereof is oxidized to yield compound (2c) or (2d) according to the following formulae or mixtures or salts thereof:

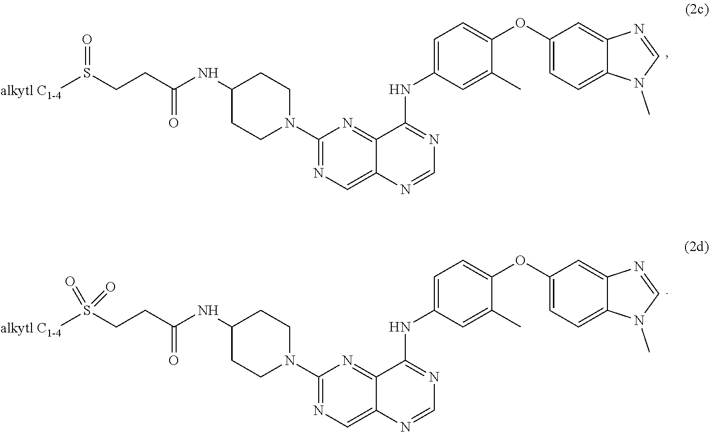

Especially leaving group comprising a sulfur atom in different oxidation states and an alkyl-group attached to said sulfur atom has been found useful for a high yield conversion to compound (1). The eliminated groups are easy to clean from the reaction mixture. Preferably this reaction can be performed in the form of a one-pot-reaction.

In an embodiment of the process, compounds (5a) or (5b) or mixtures or salts thereof, are reacted with a compound (7a) according to the following formula:

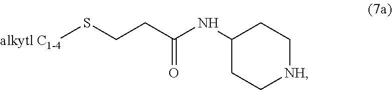

wherein the resulting reaction product compound (2b):

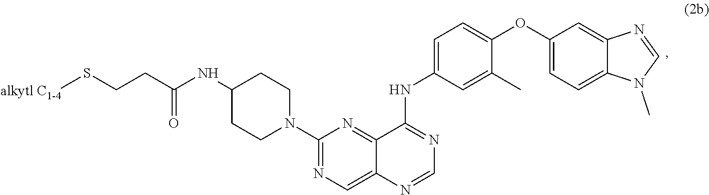

or a salt thereof is oxidized with an oxidizing agent in the presence of a solvent to yield compound (2c) or (2d) according to the following formulae or mixtures or salts thereof:

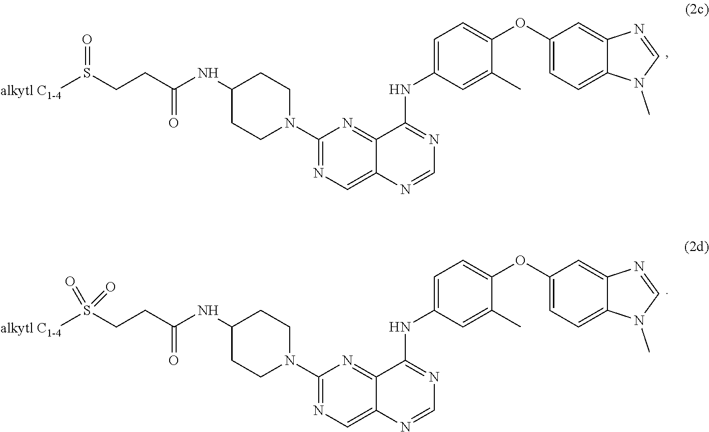

Any oxidizing agent could be used in the process according to the invention. For example, the oxidizing agent may be oxygen, a peroxide, an acid, an oxide, a dioxide, a tetroxide, a halogen, a nitrate, a sulfate, a hypochlorite, a chlorite, a chlorate, a perchlorate, a hexavalent chromium compound, a permanganate, a perborate, or a mixture thereof. Mixtures with hydrogen peroxide are preferred.

Preferably the oxidizing agent is selected from the group consisting of H₂O₂, mCPBA, ^{t}BuOOH, CH₃C(O)OH/H₂O₂, KMnO₄, MnO₂, NaMoO₄/H₂O₂, NaWO₄-2H₂O/H₂O₂ and RuCl₃/H₂O₂. Preferably the oxidizing agent is selected from the group consisting of H₂O₂, mCPBA, NaMoO₄/H₂O₂, NaWO₄, NaWO₂, NaWO₂-2H₂O/H₂O₂. Preferably the oxidizing agent is selected from the group consisting of H₂O₂, mCPBA, NaMoO₄/H₂O₂, NaWO₄-2H₂O/H₂O₂. Preferably, the oxidizing agent is mCPBA or H₂O₂. Preferably, the oxidizing agent is H₂O₂. Preferably, in the course of the oxidation a mixture of compound (2c) and (2d) is obtained, wherein said mixture comprises a larger amount of compound (2d) compared to compound (2c) in terms of percentage by weight. Preferably, said mixtures comprises up to 30% by weight of compound (2c).

In a preferred embodiment, compound (5a) or (5b) or mixtures or salts thereof are obtained by an oxidation reaction of a compound (8). Preferably, a hydrochloride salt of compound (8) is used. Preferably, a monohydrochloride or dihydrochloride salt of compound (8) is used. Preferably, the oxidizing agent is H₂O₂. Preferably, a mixture of compound (5a) and (5b) is obtained, wherein said mixture comprises a larger amount of compound (5a) compared to compound (5b) in terms of percentage by weight. Preferably, said mixture comprises compound (5a) and (5b) in a weight ratio of from 95:5 to 70:30, preferably of 85:15 to 80:20.

In a preferred embodiment, compound (8) is obtained by treating a compound (9) according to the following formula:

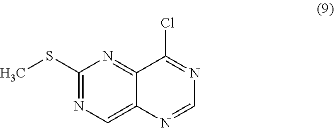

with a compound (10) according to the following formula:

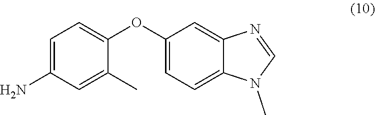 [SMILES: Cc1cc(N)ccc1Oc1ccc2c(c1)ncn2C]

or a salt thereof, in the presence of a solvent to obtain compound (8) or a salt thereof.

Preferably the solvent is toluene or IPA or a mixture thereof. Preferably, compound (8) is not isolated, such that compounds (9) and (10) can be converted to compounds (5a) and/or (5b) in two-steps performed in one-pot.

In an embodiment of the process, compound (9) according to the following formula:

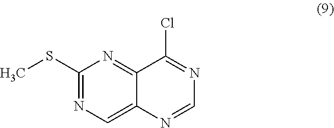

is treated with a compound (10) according to the following formula:

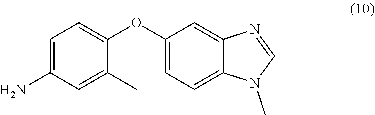 [SMILES: Cc1cc(N)ccc1Oc1ccc2c(c1)ncn2C]

or a salt thereof, in the presence of a solvent to obtain compound (8) or a salt thereof; compound (8) thus obtained is oxidized in situ to afford compound (5a) or (5b) or mixtures or salts thereof.

In an embodiment of the process, the process for the synthesis of a compound (1) as defined herein or a salt thereof, comprises the process step of reacting compound (2a) as defined herein or a salt thereof, with a base in the presence of a solvent, wherein the halogen is selected from the group consisting of Cl, Br and I.

In an embodiment of the process, X is halogen and the solvent is selected from the group consisting of water, THF, MeCN, DCM, IPA and mixtures thereof.

In an embodiment of the process, X is halogen and the base is selected from the group consisting of alkaline or alkaline earth hydroxides, alkaline or alkaline earth phosphates, alkaline or alkaline earth alkoxides, superbases, alkaline or alkaline earth carbonates, aliphatic or aromatic tertiary amines and mixtures thereof.

In an embodiment of the process, X is halogen and the base is selected from the group consisting of alkaline or alkaline earth hydroxides, alkaline or alkaline earth phosphates, alkaline or alkaline earth alkoxides, aliphatic or aromatic tertiary amines and mixtures thereof.

In an embodiment of the process, compound (2a) as depicted above is obtained by a multi-step reaction comprising at least the reaction of a compound (5a) or compound (5b) as defined herein or mixtures or salts thereof and a compound (6) according to the following formula:

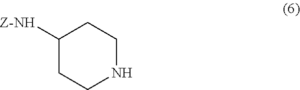

wherein Z is a protective group (PG), to result in compound (4a) as defined herein and compound (4a) is deprotected to result in compound (3) as defined herein, and compound (3) is further reacted with 3-halopropionyl halide to yield compound (2a).

In an embodiment of the process, the halogen in the multi-step process to obtain compound (2a) as explained above is Cl.

In an embodiment of the process, compound (1) is synthesized in a one-pot reaction from compound (3) in the multi-step process to obtain compound (2a) as explained above.

In an embodiment of the process, compound (2a) as depicted above is obtained by a one-step reaction comprising at least the reaction of a compound (5a) or compound (5b) as defined herein or mixtures or salts thereof and a compound (6) according to the following formula:

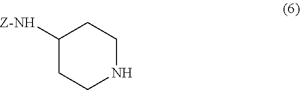

wherein Z is C═O—CH₂—CH₂-halogen, to result in compound (2a), wherein the preparation of compound (5a) or compound (5b) at least comprises the step of the preparation of a compound (8), wherein compound (8) is obtained by treating a compound (9) according to the following formula:

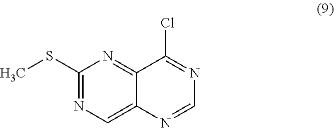

with a compound (10) according to the following formula:

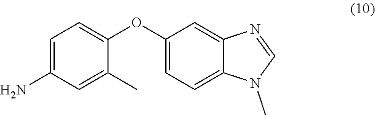 [SMILES: Cc1cc(N)ccc1Oc1ccc2c(c1)ncn2C]

or a salt thereof, in the presence of a solvent to obtain compound (8) or a salt thereof.

Preferably the solvent is toluene or IPA or a mixture thereof.

In an embodiment of the process, compound (2a) as depicted above is obtained by a multi-step reaction comprising at least the reaction of a compound (5a) or compound (5b) as defined herein or mixtures or salts thereof and a compound (6) according to the following formula:

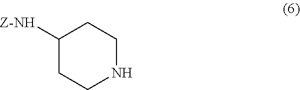

wherein Z is a protective group (PG), to result in compound (4a) as defined herein and compound (4a) is deprotected to result in compound (3) as defined herein and compound (3) is further reacted with 3-halopropionyl halide to yield compound (2a), wherein the preparation of compound (5a) or compound (5b) at least comprises the step of the preparation of a compound (8), wherein compound (8) is obtained by treating a compound (9) as defined herein with a compound (10) as defined herein or a salt thereof, in the presence of a solvent to obtain compound (8) or a salt thereof. Preferably the solvent is toluene or IPA or a mixture thereof.

In an embodiment of the process, compound (2) is selected from the group consisting of compounds (2b)-(2d) as defined herein.

In an embodiment of the process, X comprises at least one sulfur atom and the solvent is selected from the group consisting of water, THF, MeCN, DCM, IPA and mixtures thereof.

In an embodiment of the process, X comprises at least one sulfur atom and the base is selected from the group consisting of alkaline or alkaline earth hydroxides, alkaline or alkaline earth phosphates, alkaline or alkaline earth alkoxides, superbases, alkaline or alkaline earth carbonates, aliphatic or aromatic tertiary amines and mixtures thereof.

In an embodiment of the process, X comprises at least one sulfur atom and the base is selected from the group consisting of alkaline or alkaline earth hydroxides, alkaline or alkaline earth phosphates, alkaline or alkaline earth alkoxides, aliphatic or aromatic tertiary amines and mixtures thereof.

In an embodiment of the process, compound (1) is synthesized in a one-pot reaction from compound (2b).

In an embodiment of the process, compound (5a) or (5b) or mixtures thereof, are reacted with a compound (7a) as defined herein wherein the resulting reaction product compound (2b) as defined herein or a salt thereof is oxidized to yield compound (2c) or (2d) as defined herein or mixtures or salts thereof, wherein the preparation of compound (5a) or compound (5b) at least comprises the step of the preparation of a compound (8), wherein compound (8) is obtained by treating a compound (9) as defined herein with a compound (10) as defined herein or a salt thereof, in the presence of a solvent to obtain compound (8) or a salt thereof. Preferably the solvent is toluene or IPA or a mixture thereof.

A further aspect of the invention concerns compound (2) according to the following formula:

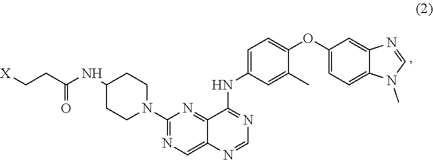

or a salt thereof, wherein X is a leaving group or a precursor of a leaving group. Compound (2) and subformulas thereof, including (2a), (2b), (2c), (2d), (2a′), (2b′), (2c′) and (2d′), can be used as intermediates in the synthesis of compound (1) as defined herein.

In an embodiment of the compound, X is selected from the group consisting of halogen, OH, sulfur comprising leaving group (such as S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), etc.), carbonate, acetate, haloacetate (such as chloroacetate) and phosphate.

In an embodiment of the compound, X is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), OC(O)C₁₋₄-alkyl, OC(O)C₁₋₄-haloalkyl, OC(O)aryl, OC(O)OC₁₋₄-alkyl, OC(O)OC₁₋₄-haloalkyl, and OC(O)Oaryl, wherein said aryl is optionally substituted by one or more substituents, each independently being the same or different C₁₋₄-alkyl.

In an embodiment of the compound, X is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), and OSO₂(C₁₋₄-haloalkyl), wherein said aryl is optionally substituted by one or more substituents, each independently being the same or different C₁₋₄-alkyl.

In an embodiment of the compound, X is selected from the group consisting of halogen, OH, S(CH₃), SO(CH₃), SO₂(CH₃), OSO₂(aryl), OC(O)OMe and OC(O)O^{t}Bu.

In an embodiment, compound (2) is a compound (2a) according to the following formula:

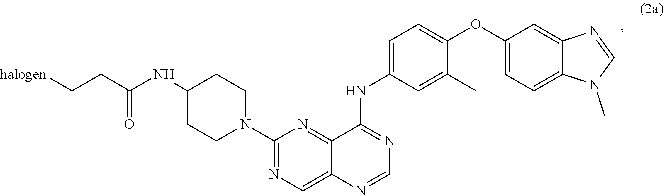

or a salt thereof. Preferably, the halogen is chlorine.

In an embodiment, compound (2) is a compound according to the following formula:

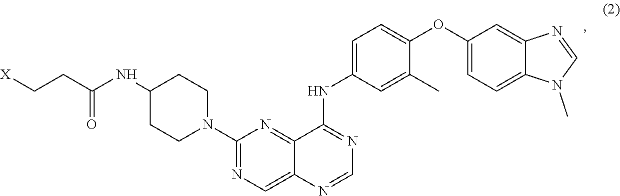

or a salt thereof, wherein X is selected from the group consisting of S(C₁₋₄-alkyl), SO(C₁₋₄-alkyl), SO₂(C₁₋₄-alkyl), OSO₂aryl, OSO₂(C₁₋₄-alkyl), and OSO₂(C₁₋₄-haloalkyl), wherein said aryl is optionally substituted by one or more substituents, each independently being the same or different C₁₋₄-alkyl.

In an embodiment, compound (2) is a compound according to the following formula:

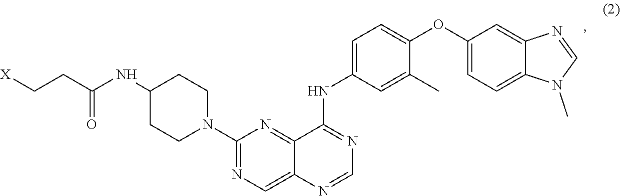

or a salt thereof, wherein X is selected from the group consisting of S(C₁₋₄-alkyl), SO(C₁₋₄-alkyl) and SO₂(C₁₋₄-alkyl).

In an embodiment, compound (2) is a compound according to the following formula:

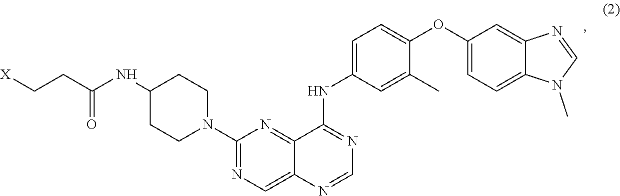

or a salt thereof, wherein X is selected from the group consisting of S(CH₃), SO(CH₃), SO₂(CH₃), OSO₂(CH₃), and OSO₂(CH₃).

In an embodiment of the compound, X is selected from the group consisting of Cl, S(CH₃), SO(CH₃) and SO₂(CH₃).

In an embodiment of the compound, compound (2) is selected from the group consisting of compounds (2a)-(2d) according to the following formulae or mixtures or salts thereof:

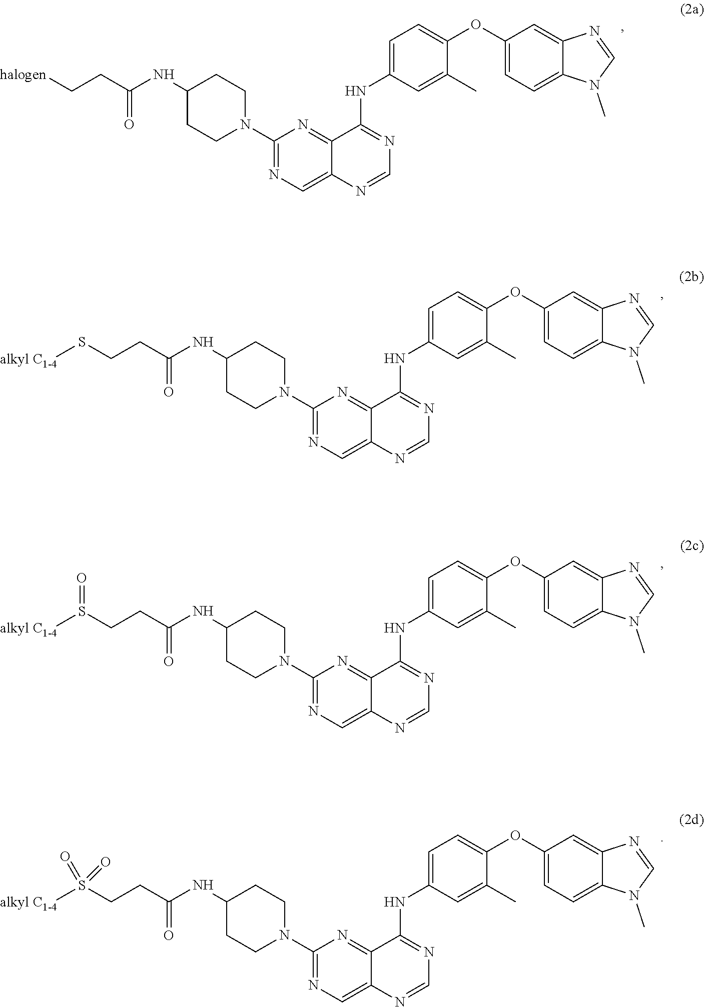

In an embodiment of the compound, compound (2) is selected from the group consisting of compounds (2a′)-(2d′) according to the following formulae or mixtures or salts thereof:

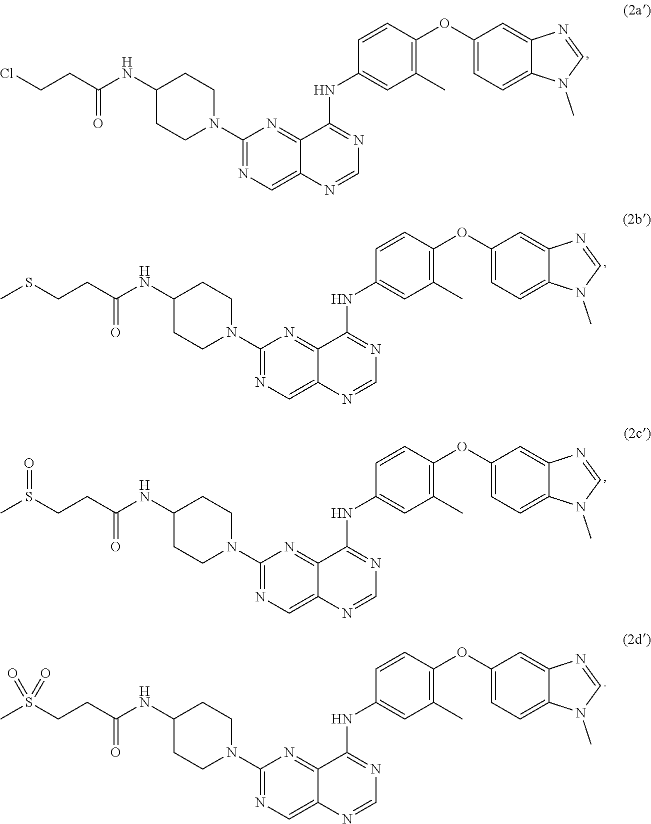 [SMILES: Cc1cc(Nc2ncnc3cnc(N4CCC(NC(=O)CCCl)CC4)nc23)ccc1Oc1ccc2c(c1)ncn2C ; CSCCC(=O)NC1CCN(c2ncc3ncnc(Nc4ccc(Oc5ccc6c(c5)ncn6C)c(C)c4)c3n2)CC1 ; Cc1cc(Nc2ncnc3cnc(N4CCC(NC(=O)CCS(C)=O)CC4)nc23)ccc1Oc1ccc2c(c1)ncn2C ; Cc1cc(Nc2ncnc3cnc(N4CCC(NC(=O)CCS(C)(=O)=O)CC4)nc23)ccc1Oc1ccc2c(c1)ncn2C]

In an embodiment, compound (2) is compound (2a′) as defined above.

In an embodiment, compound (2) is compound (2b′) as defined above.

In an embodiment, compound (2) is compound (2c′) as defined above.

In an embodiment, compound (2) is compound (2d′) as defined above.

In an embodiment, compound (2) is a salt of compound (2a′) as defined above.

In an embodiment, compound (2) is a salt of compound (2b′) as defined above.

In an embodiment, compound (2) is a salt of compound (2c′) as defined above.

In an embodiment, compound (2) is a salt of compound (2d′) as defined above.

A further aspect of the invention concerns intermediates in the synthesis of a compound (1) as defined herein or a salt thereof, wherein the intermediate is a compound according to the invention.

A further aspect of the invention concerns intermediates in the synthesis of a compound (1) as defined herein or a salt thereof, wherein the intermediate is a compound (2) according to the following formula

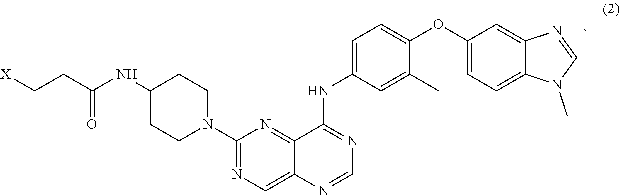

or a salt thereof, wherein X is a leaving group or a precursor of a leaving group. In other words, the invention also provides the use of a compound of formula (2) as defined above or below as an intermediate in the synthesis of compound (1).

In a preferred embodiment of the intermediate in the synthesis of a compound (1) as defined herein or a salt thereof, X is selected from the group consisting of halogen, OH, sulfur comprising leaving group (such as S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), etc.), carbonate, acetate, haloacetate (such as chloroacetate) and phosphate.

In a preferred embodiment of the intermediate in the synthesis of a compound (1) as defined herein or a salt thereof, X is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), OC(O)C₁₋₄-alkyl, OC(O)C₁₋₄-haloalkyl, OC(O)aryl, OC(O)OC₁₋₄-alkyl, OC(O)OC₁₋₄-haloalkyl, and OC(O)Oaryl, wherein said aryl is optionally substituted by one or more substituents, each independently being the same or different C₁₋₄-alkyl.

In a preferred embodiment of the intermediate in the synthesis of a compound (1) as defined herein or a salt thereof, the intermediate is a compound (2) according to the following formula

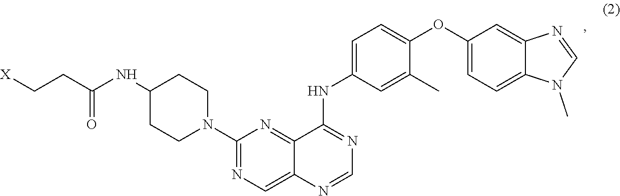

or a salt thereof, wherein X is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), and OSO₂(C₁₋₄-haloalkyl), wherein said aryl is optionally substituted by one or more substituents, each independently being the same or different C₁₋₄-alkyl.

In a preferred embodiment of the intermediate in the synthesis of a compound (1) as defined herein or a salt thereof, the intermediate is a compound (2) according to the following formula

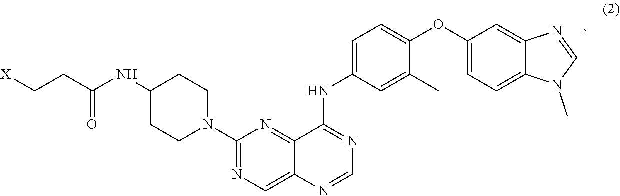

or a salt thereof, wherein X is halogen, preferably Cl.

In a preferred embodiment of the intermediate in the synthesis of a compound (1) as defined herein or a salt thereof, the intermediate is a compound (2) according to the following formula

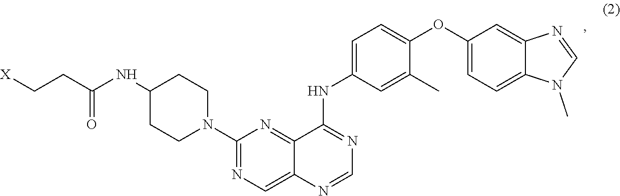

or a salt thereof, wherein X is selected from the group consisting of S(C₁₋₄-alkyl), SO(C₁₋₄-alkyl), SO₂(C₁₋₄-alkyl), OSO₂aryl, OSO₂(C₁₋₄-alkyl), and OSO₂(C₁₋₄-haloalkyl), wherein said aryl is optionally substituted by one or more substituents, each independently being the same or different C₁₋₄-alkyl.

In a preferred embodiment of the intermediate in the synthesis of a compound (1) as defined herein or a salt thereof, the intermediate is a compound (2) according to the following formula:

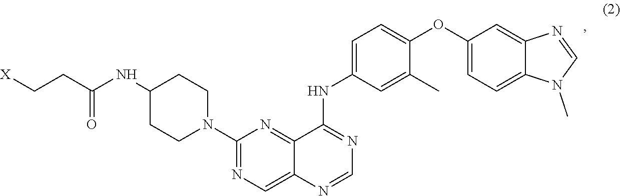

or a salt thereof, wherein X is selected from the group consisting of S(CH₃), SO(CH₃), SO₂(CH₃), OSO₂(CH₃), and OSO₂(CH₃).

Compound (1) may also be obtained by reacting compound (5a) or (5b) or mixtures or salts thereof with N-(piperidin-4-yl)acrylamide or salts thereof:

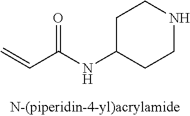

Therefore, an object of the present invention is a process for the synthesis of a compound (1) according to the following formula:

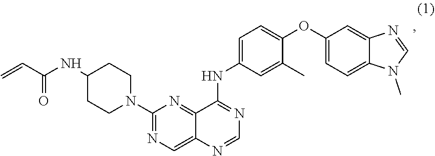 [SMILES: C=CC(=O)NC1CCN(c2ncc3ncnc(Nc4ccc(Oc5ccc6c(c5)ncn6C)c(C)c4)c3n2)CC1]

or a salt thereof, wherein the process at least comprises the process step of reacting a compound (5a) or compound (5b) according to the following formulae:

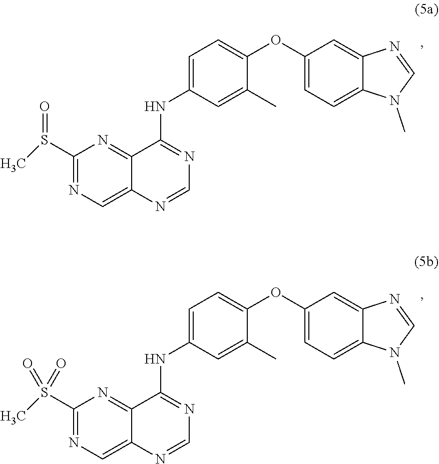 [SMILES: Cc1cc(Nc2ncnc3cnc(S(C)=O)nc23)ccc1Oc1ccc2c(c1)ncn2C ; Cc1cc(Nc2ncnc3cnc(S(C)(=O)=O)nc23)ccc1Oc1ccc2c(c1)ncn2C]

or mixtures or salts thereof, with N-(piperidin-4-yl)acrylamide:

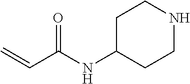

or a salt thereof.

### EXAMPLES

The examples which follow serve to illustrate the invention in more detail but do not constitute a limitation thereof.

### General

Unless stated otherwise, all reactions are carried out in commercially available apparatus using methods that are commonly used in chemical laboratories. Starting materials that are sensitive to air and/or moisture are stored under protective gas and corresponding reactions and manipulations therewith are carried out under protective gas (nitrogen or argon).

If a compound is to be represented both by a structural formula and by its nomenclature, in the event of a conflict the structural formula is decisive.

### General Schemes

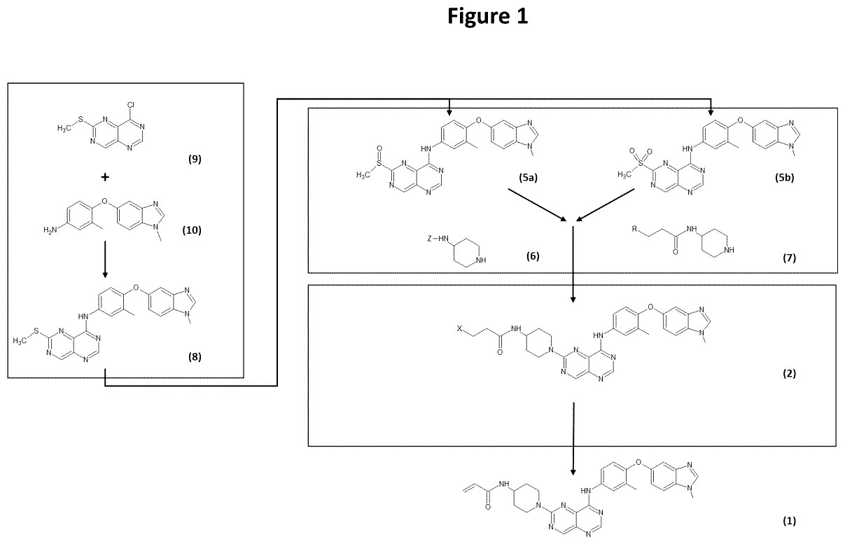

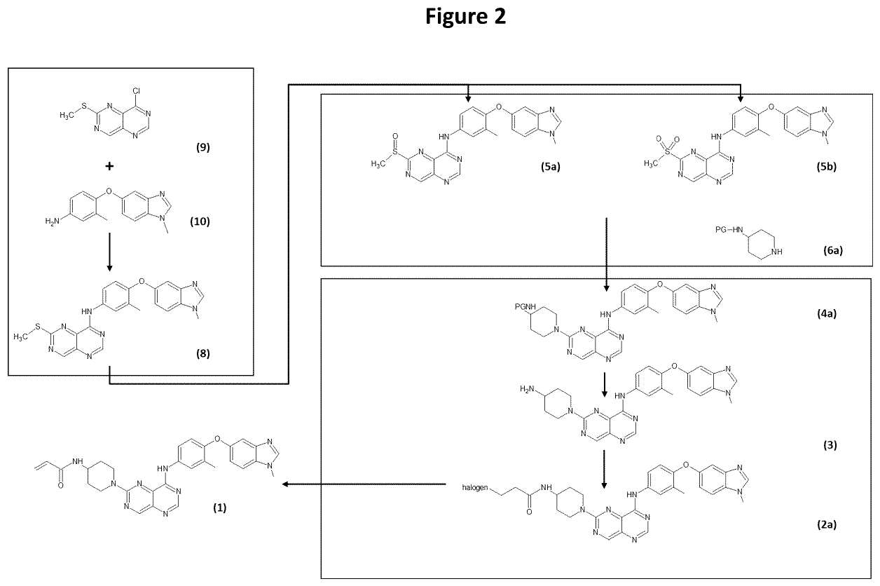

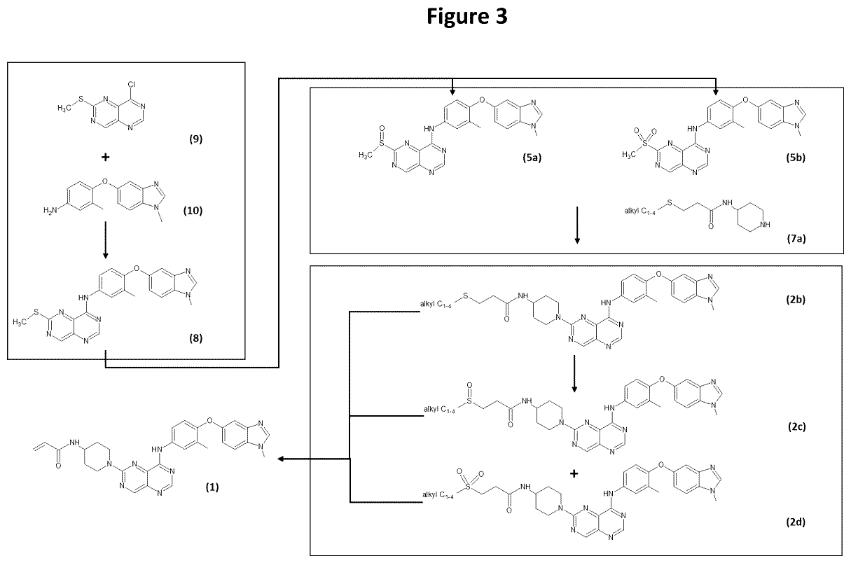

The intermediates and processes of the invention will become apparent to the one skilled in the art studying the general scheme as depicted in FIG. 1. More specific routes of synthesis are given in FIGS. 2 and 3. The figures are accompanied by the following general description of the intermediates and processes of the invention.

Starting materials (9) and (10) are commercially available and can be prepared according to procedures known in the art. For example, compound (9) can be prepared as described in WO 97/32880, WO 2010/026262 or WO 2020/239999 and compound (10) can be prepared as described in WO 2019/214634, WO 2021/156178, WO 2021/213800 or WO 2022/003575.

Compounds (9) and (10) can be reacted to afford compound (8), which can be oxidized to sulfoxide (5a) or to sulfone (5b) or a mixture thereof as reported herein or as described in WO 2021/156178 or WO 2021/213800 for the sulfoxide (5a) or in WO 2022/003575 for the sulfone (5b). Different oxidizing agents can be used, including hydrogen peroxide and m-CPBA, under a variety of conditions to favor reaction selectivity.

The oxidation of compound (8) to sulfoxide (5a) and/or sulfone (5b) may be performed in situ, i.e. without the need for its isolation. In this case, compounds (9) and (10) would lead to compounds (5a) and/or (5b) in one-pot, without the isolation of compound (8).

Reaction of compound (5a), (5b) or a mixture or salts thereof with compound (6) or (7) affords compound (2), which can in turn be reacted in the presence of a base, to yield compound (1).

Although not depicted in FIG. 1, compound (6′) as defined herein can be used in this step in place of compound (6).

Compound (5a) and/or compound (5b) may be subjected to a reaction with N-(piperidin-4-yl)acrylamide or its synthetic precursor to yield compound (1) without the need for isolation of compound (2).

With reference to FIG. 2, compound (4a) can be synthesized via SnAr reaction of commercial compound (6a), wherein PG in compound (6a) is a protective group, and sulfoxide (5a) or sulfone (5b) or a mixture or salts thereof under reaction conditions customary for an SnAr reaction. Transformation has been optimized with the Boc-protected version of compound (6a) in aqueous THF solvent systems and temperature of −60° C. to furnish product in good 80-100% isolated yields.

Compound (3) can be synthesized via deprotection of compound (4a). The exact deprotection reaction conditions depend on the identity of the PG. Removal of Boc, for instance, is preferred under a variety of acidic conditions. For example, employment of acyl chloride or sulfuric acid in iso-propyl alcohol at elevated temperatures (50-75° C.) was shown to furnish compound (3) in excellent 90-100% isolated yields.

Compound (2a) can be synthesized via coupling of compound (3) with a 3-halopropanoyl halide in the presence of a base. For example, this reaction was shown to work in aqueous THF solvent system in the presence of potassium phosphate tribasic and furnish compound (2a) in good 70-90% isolated yields.

Compound (1) can be synthesized by elimination of hydrogen halide from compound (2a) under basic conditions. Employment of aqueous KOH solution in THF allowed to achieve good conversion and obtain compound (1) in good 80-100% isolated yield.

Compound (1) can also be synthesized in one-pot from compound (3).

Turning to FIG. 3, compound (2b) can be synthesized by coupling of compounds (5a) and/or (5b) with piperidine derivative compound (7) as defined in FIG. 1, especially compound (7a) depicted in FIG. 3, in various aprotic solvents such as DMAc or 2-Me-THF or mixtures thereof. Compound (7a) can be used as a free base or a salt. Latter does not need to be subjected to free-basing prior its reaction with compounds (5a) and/or (5b). Compound (2b) is typically isolated in good 70-90% yield.

Compound (2b) can be oxidized to sulfoxide (2c) or sulfone (2d) or a mixture thereof as reported herein. Different oxidizing agents and variety of solvents can be used, including hydrogen peroxide and m-CPBA, under a variety of conditions to favor reaction selectivity.

Compound (1) can be synthesized by elimination of the sulfur comprising leaving group or precursor thereof from compound (2b), (2c), (2d) or mixtures thereof under basic conditions.

Employment of aqueous KOH solution in THF and/or acetonitrile allowed to achieve good conversion and obtain compound (1) in good 80-100% isolated yield.

Compound (1) can also be synthesized in one-pot from compound (2b), via oxidation to compound (2c), (2d) or mixtures thereof, followed by elimination.

### Analytical Methods

¹H NMR spectra were recorded in dimethyl sulfoxide-d6 (DMSO-d6) on Bruker (400 MHz) spectrometer.

MS measurements were carried out using Waters ACQUITY QDa Detector paired with LC System. Ionization Parameters were as follows: 100-800 mass range; 15V cone voltage; 15 pts/s sampling rate; 0.8V+/−capillary voltage; 600° C. probe temperature.

HPLC Method for measuring compound (5a) and compound (5b) ratio:

HPLC equipped with gradient pump (600 bar), column thermostat, UV-detector and autosampler thermostat.
Column: Waters XBridge BEH C18 4.6×100 mm, 2.5 m (or an equivalent column)
Solvents and Reagent Acetonitrile, HPLC grade
   Water, Milli-Q water or equivalent
   Ammonium formate, >99%
   Formic acid, LC-MS grade
   Methanol, HPLC grade
Buffer Preparation Accurately weigh 0.63 g of ammonium formate into a 1 L volumetric flask.
   Dissolve in 800 mL of water. Adjust pH to 2.80 using formic acid and dilute to volume with water.
   Filter the ammonium formate buffer before use.
   Assemble the filter device with a C18 filter disk (Supelco Empore or equivalent, 47 mm diameter). Install the filtration device to a waste flask.
   Make sure that the surface of the filter is covered with liquid (1-2 mm thickness) always during the activation and filtration procedure. If the filter runs dry, re-start the procedure from activation Step 1.
   Activation step 1: Pump 30-50 mL MeOH through the filter.
   Activation step 2: Pump 30-50 mL of MilliQ water through the filter.
   Filtration: Install the filtration device to a clean flask and filtrate the mobile phase.
Mobile Phase A Buffer
Mobile Phase B Acetonitrile
Diluent Acetonitrile
Flow 1.2 mL/min
Gradient

| Time, [min] | Mobile Phase B % |
|---|---|
| 0 | 10 |
| 5 | 30 |
| 15 | 50 |
| 18 | 90 |
| 18.1 | 10 |
| 20 | 10 |

Wavelength 254 nm
Column temperature 40° C.
Sample Temperature 5° C.
Retention Times:

| Component | Retention Times, [min] |
|---|---|
| Compound (5a) | 1.00 |
| Compound (5b) | 1.10 |

### Example 1—Route 1

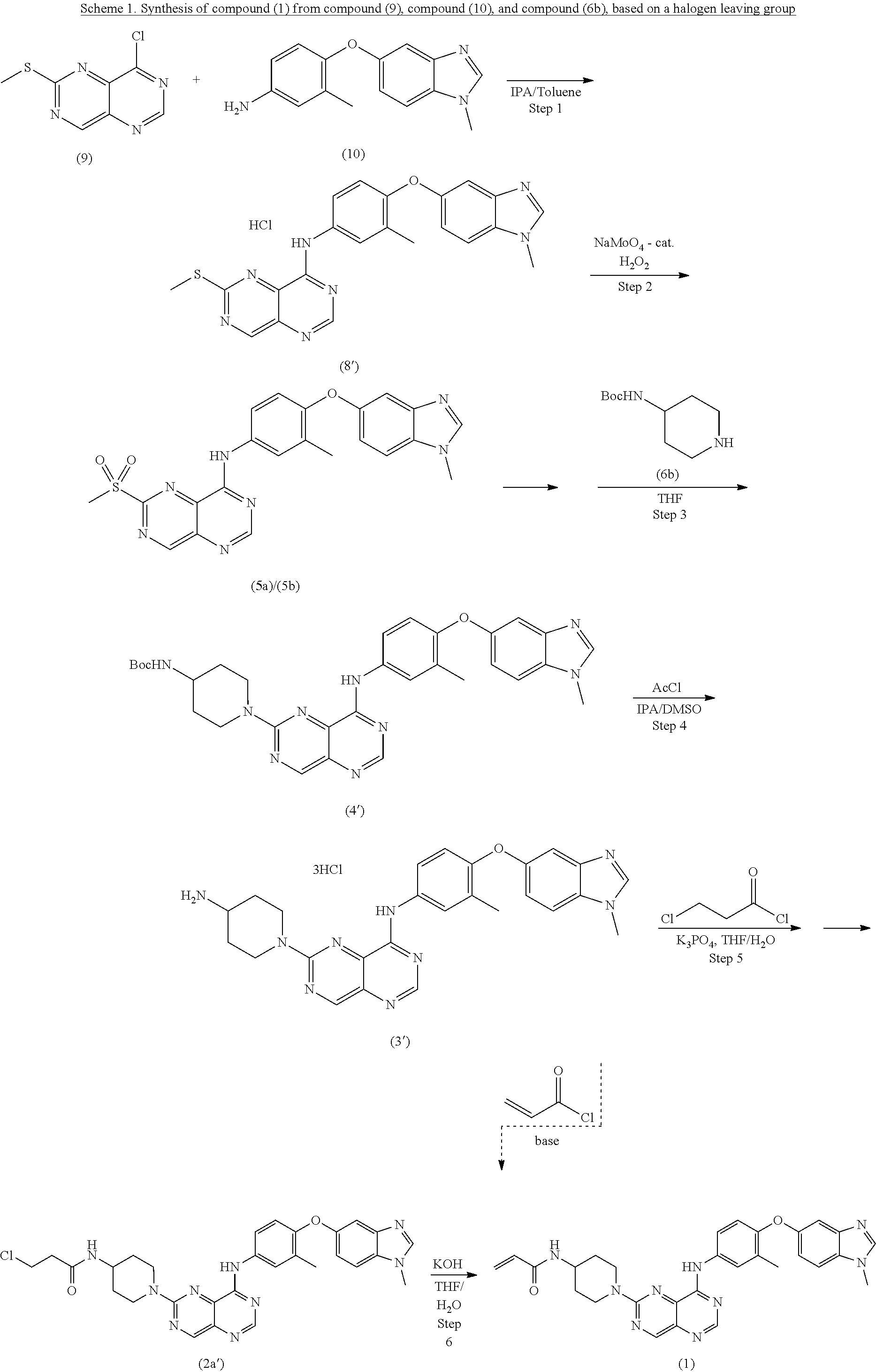

### Step 1. Preparation of Compound (8′) from Compound (9) and Compound (10)

### General Procedure

All calculations are made with respect to compound (9).

To a clean N₂-sparged vessel, compound (9) (1.0 equiv.), compound (10) (1.0 equiv.), and toluene (2.0 V) are charged. Agitation is started and IPA (10.0 V) is added. Upon completion of addition, mixture is heated to 43° C. and held until reaction is complete. Then, the reaction mixture is cooled to 22° C., agitated for 30 minutes and filtered. Solids are washed with IPA (1.5 V) twice and dried in vacuum at 50° C. to yield compound (8′) as a solid in 93% yield.

### Analytical Information

compound (8′): ¹H NMR (400 MHz, DMSO-d₆) δ ppm 2.21 (s, 3H) 2.77 (s, 3H) 4.05 (s, 3H) 7.09 (d, J=8.76 Hz, 1H) 7.15 (d, J=2.50 Hz, 1H) 7.34 (dd, J=9.01, 2.25 Hz, 1H) 7.85 (dd, J=8.63, 2.63 Hz, 1H) 7.89 (d, J=2.50 Hz, 1H) 7.97 (d, J=9.01 Hz, 1H) 8.66 (s, 1H) 9.27 (s, 1H) 9.48 (s, 1H) 10.04 (br s, 1H); LR EI MS m/z: 430.14

### Step 2. Preparation of Compound (5a)/(5b) from Compound (8′)

### General Procedure

All calculations are made with respect to compound (8′).

To a clean N₂-sparged vessel, compound (8′) (1.0 equiv., HCl salt) and ethanol (5.5 V) are charged. Agitation is started, content is sparged with N₂ and internal temperature of vessel is adjusted to 25° C. To this mixture, a solution of Na₂MoO₄ (1.1 mol %) in water (2.2 V) is charged followed by 30% aq. H₂O₂ (1.20 equiv.) charge while maintaining an internal temperature of vessel of 25° C. Upon completion of addition, mixture is agitated at 25° C. no less than 3 hours. Then, excess of H₂O₂ is quenched with Na-L-Ascorbate (0.10 equiv.) solution in water (0.3 V). Mixture is agitated for 15 minutes at 25° C. followed by addition of DMSO (6.1 V) and water (0.2 V). pH of the mixture is adjusted to 5.0-6.0 using triethylamine and the mixture is heated to 40° C. followed by addition of water (6 V) while maintaining internal temperature of vessel of 40° C. Then, mixture is brought to 25° C. and solids are filtered. Solids are washed with a solution of water (2.0 V) and Ethanol (0.5 V) and dried in vacuum at ambient temperature to yield compound (5a)/(5b) as a solid in 94% yield and 95:5 to 70:30 respected ratio as measured by the analytical method reported above.

### Analytical Information

compound (5a): ¹H NMR (400 MHz, DMSO-d₆) δ ppm 2.27 (s, 3H) 3.10 (s, 3H) 3.84 (s, 4H) 6.88 (d, J=8.76 Hz, 1H) 7.02 (dd, J=8.69, 2.19 Hz, 1H) 7.13 (d, J=2.25 Hz, 1H) 7.59 (d, J=8.75 Hz, 1H) 7.73 (dd, J=8.69, 2.56 Hz, 1H) 0.00 (d, J=6.50 Hz, 1H) 8.21 (s, 1H) 8.78 (s, 1H) 9.60 (s, 1H) 10.46 (s, 1H); LR EI MS m/z: 446.15

### Step 3. Preparation of Compound (4′) from Compound (5a)/(5b) and Compound (6b)

### General Procedure

Stoichiometry calculations of compound (6b) are based on the calculated content of compound (5a) and compound (5b). The calculated content is obtained by subtracting residual solvents, KF, ROI, and total impurities from the theoretical value of 100%, rather than comparing it to a reference standard with known potency. Calculations of THF amount are based on the weight input of the mixture of compound (5a) and compound (5b).

To a clean N₂-sparged Vessel, compound (5a)/(5b) (1.0 equiv.), compound (6b) (1.3 equiv.) and THF (9.0 V) are charged, and agitation is started. Mixture is heated to 60° C. and agitated for no less than 6 hours. Upon completion of reaction, mixture is cooled to −10° C., agitated for an hour and filtered. Wet cake is re-charged to the vessel and triturated with THF (2 V) at 5° C. for an hour, then filtered and washed with THF (1 V). Product is dried in vacuum at ambient temperature. Yield: 87%.

### Analytical Information

compound (4′): ¹H NMR (400 MHz, DMSO-d₆) δ ppm 1.40 (m, 11H) 1.86 (br d, J=10.01 Hz, 2H) 2.26 (s, 3H) 3.17 (br t, J=11.38 Hz, 2H) 3.50-3.70 (m, 2H) 3.84 (s, 3H) 4.69-5.06 (br. s., 2H) 6.86-6.95 (m, 2H) 7.00 (dd, J=8.63, 2.38 Hz, 1H) 7.10 (d, J=2.25 Hz, 1H) 7.57 (d, J=8.76 Hz, 1H) 7.78 (dd, J=8.76, 2.50 Hz, 1H) 7.84 (d, J=2.25 Hz, 1H) 8.18 (s, 1H) 8.38 (s, 1H) 9.06 (s, 1H) 9.57 (s, 1H); LR EI MS m/z: 582.25

### Step 4. Preparation of Compound (3′) from Compound (4′)

### General Procedure

All calculations are made with respect to compound (4′).

To a clean N₂-sparged Vessel #1, IPA (6.0 V) is charged, and agitation is started. Then, acetyl chloride (7.50 equiv.) is charged while maintaining internal temperature of vessel below 45° C. Mixture is heated to 65° C. and agitated for about 1 hour.

To a clean N₂-sparged Vessel #2, compound (4′) (1 equiv.) and DMSO (4.0 V) are charged, and mixture is agitated to obtain uniform slurry. The content of the Vessel #2 is charged to Vessel #1 while maintaining internal temperature of vessel at 65° C. Reaction is agitated for no less than 5 hours, then, cooled to ambient temperature and filtered. Solids are washed with IPA (2.0 V) and dried in vacuum at 50° C. to yield compound (3′) in quantitative yield.

### Analytical Information

compound (3′): ¹H NMR (400 MHz, DMSO-d₆) δ ppm 1.51-1.72 (m, 2H) 2.10 (br d, J=9.76 Hz, 2H) 2.24 (s, 3H) 3.17 (m, 2H) 3.29-3.51 (m, 1H) 4.07 (s, 3H) 4.82-5.32 (broad signal) 7.13 (d, J=8.50 Hz, 1H) 7.17 (d, J=2.25 Hz, 1H) 7.39 (dd, J=9.01, 2.25 Hz, 1H) 7.76-7.86 (m, 2H) 8.01 (d, J=9.01 Hz, 1H) 8.41 (br d, J=3.50 Hz, 3H) 8.62 (s, 1H) 9.21 (s, 1H) 9.57 (s, 1H) 10.53 (br s, 1H); LR EI MS m/z: 482.29

### Step 5. Preparation of Compound (2a′) from Compound (3′)

### General Procedure

All calculations are made with respect to compound (3′).

To a clean N₂-sparged Vessel, compound (3′) (1.0 equiv.) and THF (8.0 V) are charged and agitation is started. To the mixture, a solution of K₃PO₄-5H₂O (4.0 equiv.) in water (6.0 V) is charged while maintaining internal temperature of vessel of 22° C. Mixture is agitated until full dissolution (e.g. between 15 minutes and 1 hr depending on the scale), then, agitation is stopped, layers are settled and separated. To organic layer, a solution of K₃PO₄-5H₂O (1.02 equiv.) in water (1.5 V) is charged, agitation started, and mixture is cooled to 6° C. To this mixture a solution of 3-chloropropionyl chloride (1.02 equiv.) in anhydrous THF (0.92 V) is charged while maintaining internal temperature of 6° C. Reaction is agitated for no less than 3 hours, then, mixture is brought to ambient temperature, agitation is stopped, layers are settled and separated. To the organic layer, DCM (4.0 V) and brine are added (2.0 V). The layers are separated, and organics are filtered through a charcoal filter. The obtained solution is subjected to azeotropic distillations at atmospheric pressure to remove water targeting final volume of 8 V. The obtained slurry is brought to ambient temperature and filtered. Solids are washed with IPA (2.0 V) and dried in vacuum to obtain compound (2a′) in 69% yield.

### Analytical Information

compound (2a′): ¹H NMR (400 MHz, DMSO-d₆) δ ppm 1.33-1.51 (m, 2H) 1.89 (br dd, J=12.76, 3.00 Hz, 2H) 2.26 (s, 3H) 2.58 (t, J=6.38 Hz, 2H) 3.20-3.32 (m, 2H) 3.76-3.87 (m, 5H) 3.88-4.02 (m, 1H) 4.83 (br signal, 2H) 6.89 (d, J=8.76 Hz, 1H) 7.00 (dd, J=8.76, 2.25 Hz, 1H) 7.10 (d, J=2.25 Hz, 1H) 7.57 (d, J=8.76 Hz, 1H) 7.77 (dd, J=8.76, 2.50 Hz, 1H) 7.84 (d, J=2.50 Hz, 1H) 8.04 (d, J=7.50 Hz, 1H) 8.17 (s, 1H) 8.39 (s, 1H) 9.07 (s, 1H) 9.58 (s, 1H); LR EI MS m/z: 572.17

### Step 6. Preparation of Compound (1) from Compound (2a′)

### General Procedure

All calculations are made with respect to compound (2a′).

To a clean N₂-sparged Vessel, compound (2a′) (1.0 equiv.) and THF (8.0 V) are charged and agitation is started. To this mixture, a solution of KOH (2.0 equiv.) in water (3.5 V) is charged while maintaining internal temperature of vessel of 23° C. Reaction is heated to 38° C. and agitated for no less then 18 hours. Upon completion of reaction, mixture is brought to ambient temperature, agitation is stopped, and aqueous layer is removed. Organics are concentrated under vacuum to target volume of 4.0 V and diluted with DCM (6.0 V) and MeOH (3.0 V). Resulting solution is washed with brine (2.0 V) and organics are separated. DCM and MeOH are exchanged with THF via atmospheric distillation to target volume of 8.0 V. Mixture is brought to ambient temperature and slurry is filtered. Solids are washed with THF (2.0 V) and dried in vacuum to yield compound (1) in 91% yield.

One-Pot Procedure for the Preparation of Compound (1) from Compound (3′).

All calculations are made with respect to compound (3′).

To a clean N₂-sparged Vessel, compound (3′) (1.0 equiv.) and THF (8.0 V) are charged, and agitation is started. To the mixture, a solution of K₃PO₄-5H₂O (4.0 equiv.) in water (6.0 V) is charged while maintaining internal temperature of vessel of 22° C. Mixture is agitated until full dissolution (e.g. between 15 minutes and 1 hr depending on the scale), then, agitation is stopped, layers are settled and separated. To organic layer, a solution of K₃PO₄-5H₂O (1.02 equiv.) in water (1.5 V) is charged, agitation started, and mixture is cooled to 6° C. To this mixture a solution of 3-chloropropionyl chloride (1.02 equiv.) in anhydrous THF (0.92 V) is charged while maintaining internal temperature of 6° C. Reaction is agitated for no less than 3 hours, then, mixture is brought to ambient temperature, agitation is stopped, layers are settled and separated. To the organic layer, DCM (4.0 V) and brine are added (2.0 V). The layers are separated, and organics are filtered through a charcoal filter. DCM is removed via distillations, and volume is adjusted to 8V with addition of THF. To this mixture, a solution of KOH (2.0 equiv.) in water (3.5 V) is charged while maintaining internal temperature of vessel of 23° C. Reaction is heated to 38° C. and agitated for no less than 18 hours. Upon completion of reaction, mixture is brought to ambient temperature, agitation is stopped, and aqueous layer is removed. Organics are concentrated under vacuum to target volume of 4.0 V and diluted with DCM (6.0 V) and MeOH (3.0 V). Resulting solution is washed with brine (2.0 V) and organics are separated. DCM and MeOH are exchanged with THF via atmospheric distillation to target volume of 8.0 V. Mixture is brought to ambient temperature and slurry is filtered. Solids are washed with THF (2.0 V) and dried in vacuum to yield compound (1).

### Example 2—Route 2

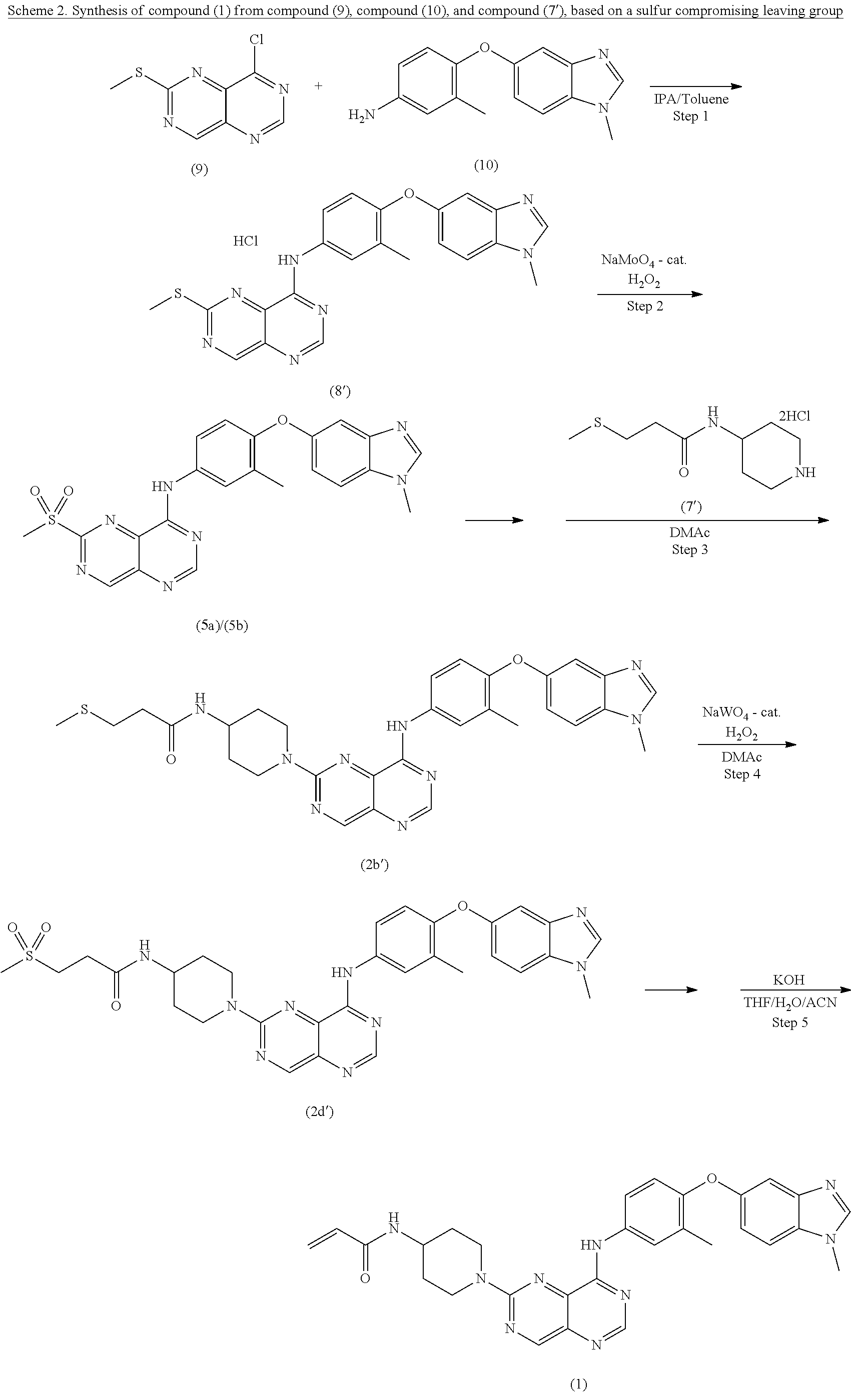

Steps 1 and 2 are performed as described in Example 1.

### Step 3. Preparation of Compound (2b′) from Compound (5a)/(5b) and Compound (7′)

### General Procedure

All calculations are made with respect to compound (5a).

To a clean N₂-sparged Vessel #1, compound (7′) (1.4 equiv.), water (1.0 V), brine (0.75 V), and 2-Me-THF (9.0 V) are charged and agitation is started. Mixture is cooled to 10° C. and 50% aqueous NaOH solution (0.47 V) is added while maintaining internal temperature of vessel below 20° C. Biphasic mixture is vigorously agitated. Agitation is stopped after layers form or after approximately 30 to 60 minutes, layers are settled and separated. Aqueous layer is back-extracted with 2-Me-THF (4 V). Then, organics are combined and 2-Me-THF is exchanged with DMAc to target volume of ˜7 V. Solution of free base of compound (7′) in DMAc thus obtained is stored until used in the next steps.

To a clean N₂-sparged Vessel #2, compound (5a)/(5b) (1.0 equiv.) is charged followed by addition of compound (7′) solution in DMAc. Agitation is started, mixture is heated to 68° C. and held for approximately 6 hours. Then, mixture is brought to ambient temperature and filtered. Solids are washed with MeOH (4 V) and dried in vacuum to yield compound (2b′) as a solid in 76% yield.

### Analytical Information

compound (2b′): ¹H NMR (400 MHz, DMSO-d₆) δ ppm 1.33-1.49 (m, 1H) 1.83-1.95 (m, 1H) 2.06 (s, 1H) 2.26 (s, 1H) 2.37 (t, J=7.25 Hz, 1H) 2.67 (t, J=7.25 Hz, 1H) 3.26 (br t, J=11.51 Hz, 1H) 3.84 (s, 1H) 3.88-3.98 (m, 1H) 4.75-4.90 (m, 1H) 6.89 (d, J=8.75 Hz, 1H) 7.00 (dd, J=8.50, 2.00 Hz, 1H) 7.10 (d, J=2.00 Hz, 1H) 7.57 (d, J=8.76 Hz, 1H) 7.77 (dd, J=8.76, 2.25 Hz, 1H) 7.84 (d, J=2.00 Hz, 1H) 7.91 (br d, J=7.50 Hz, 1H) 8.17 (s, 1H) 8.39 (s, 1H) 9.07 (s, 1H) 9.57 (s, 1H); LR EI MS m/z: 292.74.

### Step 4. Preparation of Compound (2d′) from Compound (2b′)

### General Procedure

All calculations are made with respect to compound (2b′).

To a clean N₂-sparged Vessel #1, compound (2b′) (1.0 equiv.), DMAc (8.0 V), and a solution of NaWO₄ (2 mol %) in water (0.45 V) are charged. Agitation is started and mixture is heated to 70° C. 30% aqueous solution of H₂O₂ (2.6 equiv.) is charged slowly while maintaining internal temperature of vessel of 70° C. Upon completion of addition, mixture is agitated at 70° C. for approximately 16 hours, then, cooled to 50° C. and DMSO (0.22 equiv.) is added. Mixture is further cooled to 4° C. followed by addition of IPAc (4.0 V). The obtained slurry is agitated for an additional 4 hrs at 4° C. and filtered. Solids are washed with a mixture of DMAc (0.76 V) and IPAc (2.25V) and dried in vacuum. Obtained compound (2d′) is recharged into the vessel and triturated in water (16 V) at 72° C. for 18 hours. Then, slurry is cooled, product is filtered and dried in vacuum.

If needed, compound (2c′) can be enriched in reaction mixture with employment of sub-stoichiometric amount of oxidant and then isolated by standard purification methods. Compound (2c′) is characterized below on 300 MHz NMR spectrometer and infusion MS.

### Analytical Information

compound (2d′): ¹H NMR (400 MHz, DMSO-d₆) δ ppm 1.34-1.48 (m, 2H) 1.85-1.95 (m, 2H) 2.54-2.62 (m, 2H) 2.99 (s, 3H) 3.22-3.38 (m, 4H) 3.84 (s, 3H) 3.87-4.00 (m, 1H) 4.82 (br d, 2H) 6.89 (d, J=8.75 Hz, 1H) 7.00 (dd, J=8.76, 2.25 Hz, 1H) 7.10 (d, J=2.25 Hz, 1H) 7.57 (d, J=8.50 Hz, 1H) 7.77 (dd, J=8.76, 2.75 Hz, 1H) 7.84 (d, J=2.50 Hz, 1H) 8.10 (d, J=7.75 Hz, 1H) 8.17 (s, 1H) 8.39 (s, 1H) 9.06 (s, 1H) 9.56 (s, 1H); LR EI MS m/z: 616.22.

compound (2c′): ¹H NMR (300 MHz, DMSO-d₆) δ ppm 1.34-1.58 (m, 2H) 1.83-2.02 (m, 2H) 2.27 (s, 3H) 2.50-2.66 (m, 4H) 2.77-3.41 (m, 6H) 3.84 (s, 3H) 3.88-4.05 (m, 1H) 4.77 (br d, J=13.33 Hz, 1H) 6.90 (d, J=8.65 Hz, 1H) 6.99 (dd, J=8.72, 2.27 Hz, 1H) 7.13 (d, J=2.20 Hz, 1H) 7.53 (d, J=8.79 Hz, 1H) 7.73-7.85 (m, 2H) 7.94 (d, J=7.47 Hz, 1H) 8.13 (s, 1H) 8.38 (s, 1H) 9.02 (s, 1H) 9.37 (s, 1H); LR MS: 600.25.

### Step 5. Preparation of Compound (1) from Compound (2d′)

### General Procedure

All calculations are made with respect to compound (2d′).

To a clean N₂-sparged Vessel #1, compound (2d′) (1.0 equiv.), 45% aqueous KOH solution (1.5 equiv.), THF (8.9 V), and MeCN (1.6 V) are charged and agitation is started. Mixture is heated to 42° C. and agitated for ˜14 hrs followed by cooling to 30° C. and agitation for an additional 10 hrs. Upon completion of reaction, mixture is reheated to 45° C., agitation is stopped, and layers are separated. The organic layer is consecutively washed with 2.5 M Phosphate Buffer and brine. Organics are subjected to azeotropic distillation using THF to remove water targeting final 6 V volume. Upon completion of distillation, slurry is brought to ambient temperature, filtered and washed with THF. Solids are dried in vacuum at 50° C. to yield compound (1) in 81% yield.

### Alternative Procedure

All calculations are made with respect to compound (2d′).

To a clean N₂-sparged Vessel, compound (2d′) (1.0 equiv.), 45% aqueous KOH solution (1.5 equiv.), MeCN (5.5 V), and H₂O (1.0V) are charged and agitation is started. Mixture is heated to 55° C. and agitated for ˜45 mins followed by addition of THF (3.0V). Then, mixture is agitated at 55° C. for additional 6 hours, then, cooled to ˜5° C. Slurry is agitated at this temperature for additional 2 hours and filtered. Solids are washed with mixture of 9:1=MeCN:H₂O and dried in vacuum at 50° C. to yield crude compound (1) in ˜70% yield.

1. Process for the synthesis of a compound (1) according to the following formula: 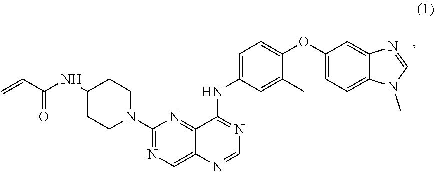 [SMILES: C=CC(=O)NC1CCN(c2ncc3ncnc(Nc4ccc(Oc5ccc6c(c5)ncn6C)c(C)c4)c3n2)CC1]
or a salt thereof, wherein the process at least comprises the process step of reacting a compound (2) according to the following formula: 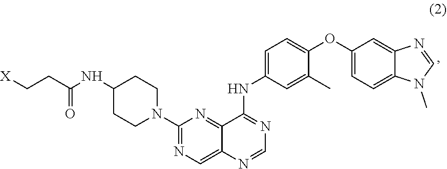
or a salt thereof, with a base in the presence of a solvent, wherein X is a leaving group or a precursor of a leaving group.

2. The process according to claim 1, wherein X is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), OCO(C₁₋₄-alkyl), OCO(C₁₋₄-haloalkyl), OCOaryl, OC(O)OC₁₋₄-alkyl, OC(O)OC₁₋₄-haloalkyl, and OC(O)Oaryl, wherein said aryl is optionally substituted by one or more substituents, each independently being the same or different C₁₋₄-alkyl.

3. The process according to claim 1, wherein X is selected from the group consisting of halogen, S(C₁₋₄-alkyl), SO(C₁₋₄-alkyl) and SO₂(C₁₋₄-alkyl).

4. The process according to claim 1, wherein compound (2) is selected from the group consisting of compounds (2a)-(2d) according to the following formulae or mixtures or salts thereof:

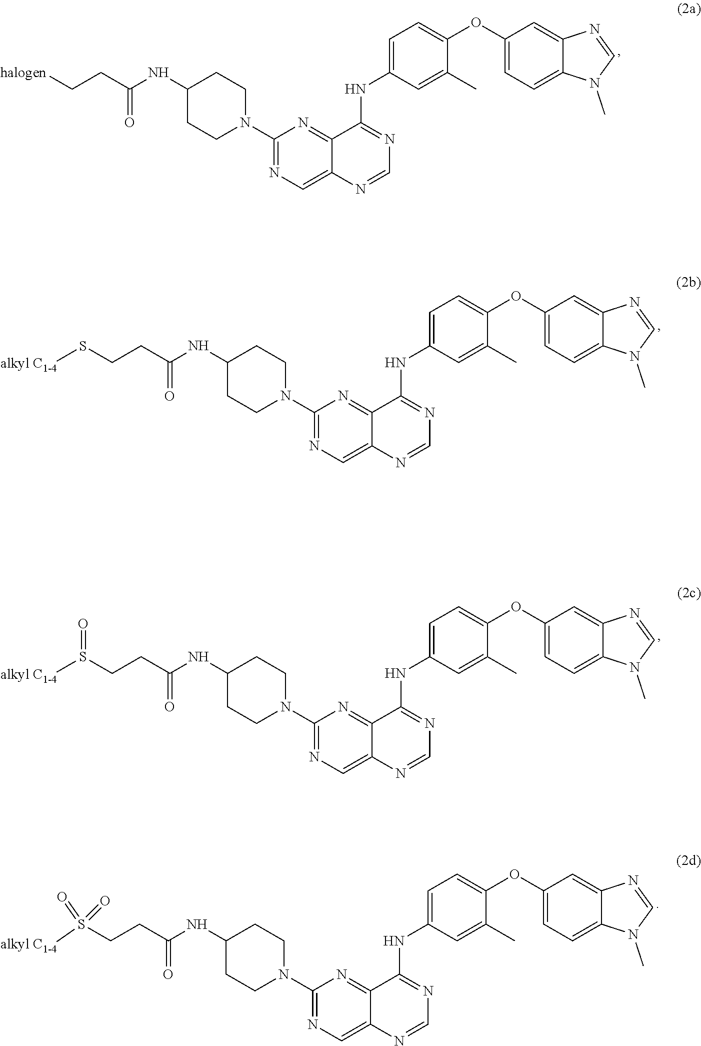

5. The process according to claim 1, wherein the solvent is selected from the group consisting of water, THF, Me-THF, MeCN, DCE, DCM, IPA, NMP, DMAc, DMF, EtOH, MTBE and mixtures thereof.

6. The process according to claim 1, wherein the solvent is selected from the group consisting of THF, MeCN, a mixture of THF and MeCN, a mixture of water and THF, a mixture of water and MeCN and a mixture of water, THF and MeCN.

7. The process according to claim 1, wherein the base is selected from the group consisting of alkaline or alkaline earth hydroxides, alkaline or alkaline earth phosphates, alkaline or alkaline earth alkoxides, superbases, alkaline or alkaline earth carbonates, aliphatic or aromatic tertiary amines and mixtures thereof.

8. The process according to claim 1, wherein the base is selected from the group consisting of KOH, NaOH, K₃PO₄, K₂CO₃, DBU, DABCO, potassium t-butoxide and Et₃N.

9. The process according to claim 1, wherein the base is potassium hydroxide.

10. The process according to claim 1, wherein X is halogen and compound (2a) is obtained by a one- or multi-step reaction comprising at least the reaction of a compound (5a) or compound (5b) according to the following formulae: 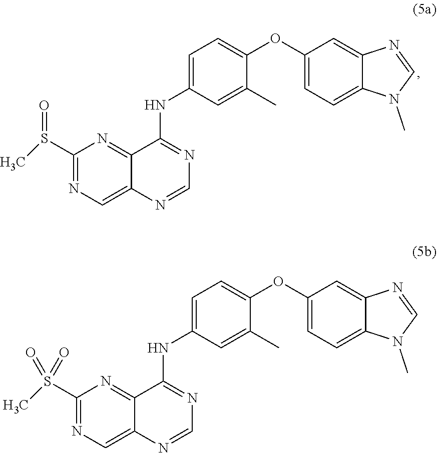 [SMILES: Cc1cc(Nc2ncnc3cnc(S(C)=O)nc23)ccc1Oc1ccc2c(c1)ncn2C ; Cc1cc(Nc2ncnc3cnc(S(C)(=O)=O)nc23)ccc1Oc1ccc2c(c1)ncn2C]
or mixtures or salts thereof and a compound (6′) according to the following formula: 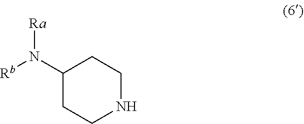
wherein R^{a} and R^{b} are each independently selected from the group consisting of hydrogen, C═O—CH₂—CH₂-halogen and a protective group (PG), wherein if both R^{a} and R^{b} are a protective group (PG), R^{a} and R^{b} together with the nitrogen atom they are bound to optionally form a ring, provided that at least one of R^{a} and R^{b} is not hydrogen, to result in compound (4b) according to the following formula:

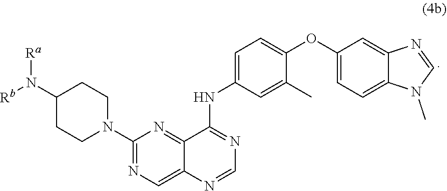

11. The process according to claim 10, wherein compound (6′) is a compound (6) according to the following formula: 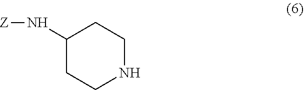
wherein Z is a protective group (PG) or C═O—CH₂—CH₂-halogen, such that the one or multi-step reaction results in compound (4) according to the following formula:

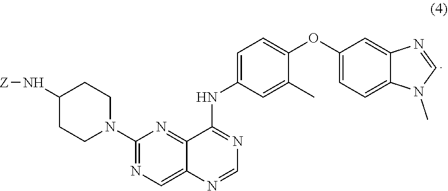

12. The process according to claim 11, wherein Z is a protective group (PG) and compound (4) is a compound (4a) according to the following formula: 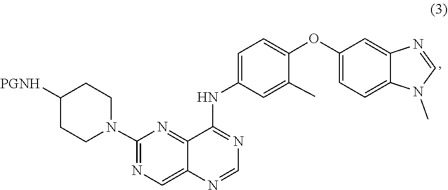
and compound (4a) is deprotected to result in compound (3) according to the following formula: 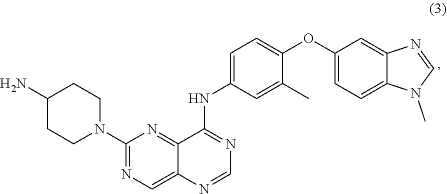 [SMILES: Cc1cc(Nc2ncnc3cnc(N4CCC(N)CC4)nc23)ccc1Oc1ccc2c(c1)ncn2C]
and compound (3) is further reacted with 3-halopropionyl halide to yield compound (2a):

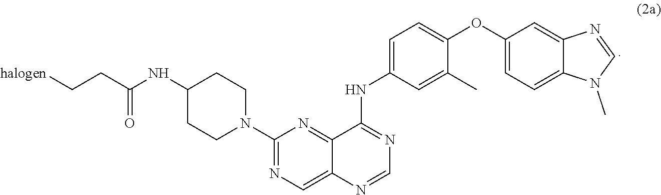

13. The process according to claim 2, wherein X is Cl.

14. The process according to claim 1, wherein compound (2) is obtained by a one- or multi-step reaction comprising at least the reaction of a compound (5a) or a compound (5b) according to the following formulae: 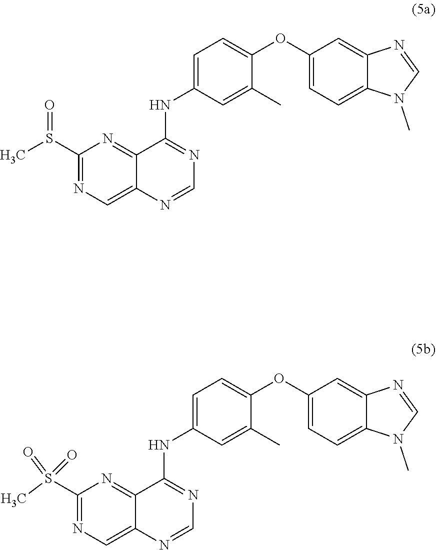 [SMILES: Cc1cc(Nc2ncnc3cnc(S(C)=O)nc23)ccc1Oc1ccc2c(c1)ncn2C ; Cc1cc(Nc2ncnc3cnc(S(C)(=O)=O)nc23)ccc1Oc1ccc2c(c1)ncn2C]
or mixtures or salts thereof and a compound (7) according to the following formula: 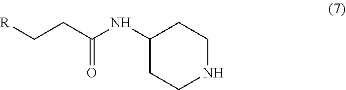
or a salt thereof, wherein R is a leaving group or the precursor of a leaving group.

15. The process according to claim 14, wherein R is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), OCO(C₁₋₄-alkyl), OCO(C₁₋₄-haloalkyl), OCOaryl, OC(O)OC₁₋₄-alkyl, OC(O)OC₁₋₄-haloalkyl, and OC(O)Oaryl, wherein said aryl is optionally substituted by one or more substituents, each independently being the same or different C₁₋₄-alkyl.

16. The process according to claim 14, wherein R comprises at least one sulfur atom.

17. The process according to claim 14, wherein R is selected from the group consisting of S(C₁₋₄-alkyl), SO(C₁₋₄-alkyl) and SO₂(C₁₋₄-alkyl).

18. The process according to claim 14, wherein compounds (5a) or (5b) or mixtures or salts thereof, are reacted with a compound (7a) according to the following formula: 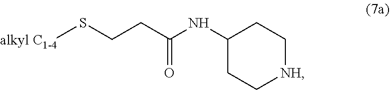
wherein the resulting reaction product compound (2b): 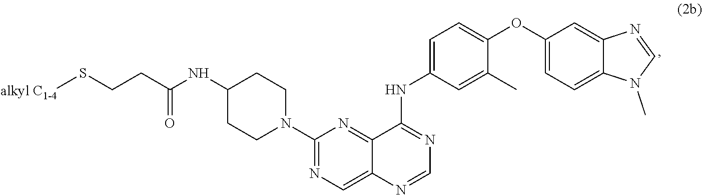
or a salt thereof is oxidized to yield compound (2c) or (2d) according to the following formulae or mixtures or salts thereof:

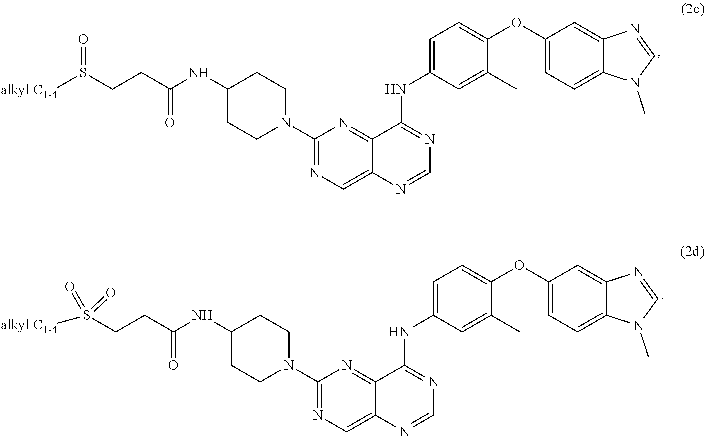

19. The process according to claim 1, wherein compound (1) is synthesized in a one-pot reaction from compound (3) or from compound (2b).

20. A compound (2) according to the following formula: 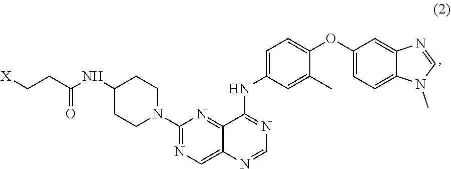
or a salt thereof, wherein X is a leaving group or a precursor of a leaving group.

21. The compound or salt according to claim 20, wherein X is selected from the group consisting of halogen, OH, S(C₁₋₄-alkyl), SO(C₁₋₄-alkyl), SO₂(C₁₋₄-alkyl), OSO₂aryl, OSO₂(C₁₋₄-alkyl), OSO₂(C₁₋₄-haloalkyl), OCO(C₁₋₄-alkyl), OCO(C₁₋₄-haloalkyl), OCOaryl, OC(O)OC₁₋₄-alkyl, OC(O)OC₁₋₄-haloalkyl, and OC(O)Oaryl, wherein said aryl is optionally substituted by one or more substituents, each independently being the same or different C₁₋₄-alkyl.

22. The compound or salt according to claim 20, wherein X is selected from the group consisting of Cl, S(CH₃), SO(CH₃) and SO₂(CH₃).
